SEQUENCE LISTING (23 sequences)

SEQ ID NO: 1 - DNA, 281 bp, synthetic construct
  misc_feature 1..281: note = R6K gamma origin
  source 1..281: mol_type = other DNA
ggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatc

SEQ ID NO: 2 - DNA, 281 bp, synthetic construct
  misc_feature 1..281: note = 1 CpG R6K gamma origin
  source 1..281: mol_type = other DNA
ggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacatactatcaacaggttgaactgctgatc

SEQ ID NO: 3 - DNA, 260 bp, synthetic construct
  misc_feature 1..260: note = CpG free R6K gamma origin
  source 1..260: mol_type = other DNA
aaaccttaaaacctttaaaagccttatatattcttttttttcttataaaacttaaaaccttagaggctatttaagttgctgatttatattaattttattgttcaaacatgagagcttagtacatgaaacatgagagcttagtacattagccatgagagcttagtacattagccatgagggtttagttcattaaacatgagagcttagtacattaaacatgagagcttagtacatactatcaacaggttgaactgctgatc

SEQ ID NO: 4 - DNA, 389 bp, synthetic construct
  misc_feature 1..389: note = Extended R6K gamma origin
  source 1..389: mol_type = other DNA
tgtcagccgttaagtgttcctgtgtcactgaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatc

SEQ ID NO: 5 - DNA, 139 bp, synthetic construct
  misc_feature 1..139: note = RNA-OUT selectable marker
  source 1..139: mol_type = other DNA
gtagaattggtaaagagagtcgtgtaaaatatcgagttcgcacatcttgttgtctgattattgatttttggcgaaaccatttgatcatatgacaagatgtgtatctaccttaacttaatgattttgataaaaatcatta

SEQ ID NO: 6 - DNA, 69 bp, synthetic construct
  misc_feature 1..69: note = RNA-OUT antisense repressor RNA
  source 1..69: mol_type = other DNA
tcgcacatcttgttgtctgattattgatttttggcgaaaccatttgatcatatgacaagatgtgtatct

SEQ ID NO: 7 - DNA, 139 bp, synthetic construct
  misc_feature 1..139: note = 2 CpG RNA-OUT selectable marker
  source 1..139: mol_type = other DNA
gtagaattggtaaagagagttgtgtaaaatattgagttcgcacatcttgttgtctgattattgatttttggcgaaaccatttgatcatatgacaagatgtgtatctaccttaacttaatgattttgataaaaatcatta

SEQ ID NO: 8 - DNA, 466 bp, synthetic construct
  misc_feature 1..466: note = R6K gamma origin -RNA-OUT bacterial replication-selection region flanked by NheI and KpnI restriction sites
  source 1..466: mol_type = other DNA
gctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacc

SEQ ID NO: 9 - DNA, 439 bp, synthetic construct
  misc_feature 1..439: note = 1 CpG R6K gamma origin - 2 CpG RNA-OUT bacterial replication-selection region flanked by NheI and KpnI restriction sites
  source 1..439: mol_type = other DNA
gctagctggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattaggtacc

SEQ ID NO: 10 - DNA, 565 bp, synthetic construct
  misc_feature 1..565: note = pNTC-NP1 polylinker trpA R6K-RNA-OUT polylinker cloning cassette: EcoRI-HindIII
  source 1..565: mol_type = other DNA
gaattcgagctcggtacctcgcgaatgcatctaggggacggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggagctagcattgggtcatcggatcccgggcccgtcgactgcagaggcctgcatgcaagctt

SEQ ID NO: 11 - DNA, 584 bp, synthetic construct
  misc_feature 1..584: note = pNTC-NP2 polylinker trpA R6K-RNA-OUT polylinker cloning cassette: EcoRI-HindIII
  source 1..584: mol_type = other DNA
gaattcgagctcggtacctcgcgaatgcatctaggggacggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggactagtcccgggcgctagttattaatattgggtcatcggatcccgggcccgtcgactgcagaggcctgcatgcaagctt

SEQ ID NO: 12 - DNA, 557 bp, synthetic construct
  misc_feature 1..557: note = pNTC-NP3 polylinker trpA R6K-RNA-OUT polylinker cloning cassette: EcoRI-HindIII
  source 1..557: mol_type = other DNA
gaattcgagctcggtacctcgcgaatgcatctaggggacggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccgagctcggatcccgggcccgtcgactgcagaggcctgcatgcaagctt

SEQ ID NO: 13 - DNA, 557 bp, synthetic construct
  misc_feature 1..557: note = pNTC-NP4 polylinker trpA R6K-RNA-OUT polylinker cloning cassette: HindIII-EcoRI
  source 1..557: mol_type = other DNA
aagcttgcatgcaggcctctgcagtcgacgggcccgggatccgagctcggtacctcgcgaatgcatctaggggacggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccgagctcgaattc

SEQ ID NO: 14 - DNA, 503 bp, synthetic construct
  misc_feature 1..503: note = pNTC-NP5 polylinker trpA R6K-RNA-OUT polylinker cloning cassette: KasI-HindIII
  source 1..503: mol_type = other DNA
ggcgccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccacatgtcctgcagaggcctgcatgcaagctt

SEQ ID NO: 15 - DNA, 539 bp, synthetic construct
  misc_feature 1..539: note = pNTC-NP6 polylinker trpA R6K-RNA-OUT polylinker cloning cassette: EcoRI-SacI
  source 1..539: mol_type = other DNA
gaattctgcagatatccatcacactggcggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccgggccccccctcgatcgaggtcgacggtatcgggggagctc

SEQ ID NO: 16 - DNA, 500 bp, synthetic construct
  misc_feature 1..500: note = pNTC-NP7 polylinker trpA R6K-RNA-OUT polylinker cloning cassette: BssHII-BssHII
  source 1..500: mol_type = other DNA
gcgcgcagccttaattaagctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccttaattaactgcgcgc

SEQ ID NO: 17 - DNA, 530 bp, synthetic construct
  misc_feature 1..530: note = pNTC-3xCpG NP1 polylinker R6K-RNA-OUT polylinker cloning cassette: HindIII-EcoRI
  source 1..530: mol_type = other DNA
aagcttgcatgcaggcctctgcagtcgacgggccctctagactcgagctggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattaggtaccgctagcggccgtcccctagatgcattcgcgaggtaccgagctcgaattc

SEQ ID NO: 18 - DNA, 303 bp, synthetic construct
  misc_feature 1..303: note = R6K gamma origin -7 iteron
  source 1..303: mol_type = other DNA
ggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgttaaacatgagagcttagtacgtactatcaacaggttgaactgctgatc

SEQ ID NO: 19 - DNA, 22 bp, synthetic construct
  misc_feature 1..22: note = R6K gamma origin iteron
  source 1..22: mol_type = other DNA
aaacatgagagcttagtacgtg

SEQ ID NO: 20 - DNA, 22 bp, synthetic construct
  misc_feature 1..22: note = R6K gamma origin iteron
  source 1..22: mol_type = other DNA
aaacatgagagcttagtacgtt

SEQ ID NO: 21 - DNA, 22 bp, synthetic construct
  misc_feature 1..22: note = R6K gamma origin iteron
  source 1..22: mol_type = other DNA
agccatgagagcttagtacgtt

SEQ ID NO: 22 - DNA, 22 bp, synthetic construct
  misc_feature 1..22: note = R6K gamma origin iteron
  source 1..22: mol_type = other DNA
agccatgagggtttagttcgtt

SEQ ID NO: 23 - DNA, 22 bp, synthetic construct
  misc_feature 1..22: note = R6K gamma origin iteron
  source 1..22: mol_type = other DNA
aaacatgagagcttagtacgta

(19) United States
(12) Patent Application Publication
(10) Pub. No.: US 2026/0286384 A1
(43) Pub. Date: Sep. 24, 2026

(54) VIRAL AND NON-VIRAL NANOPLASMID VECTORS WITH IMPROVED PRODUCTION

(71) Applicant: Aldevron, L.L.C., Fargo, ND (US)

(72) Inventor: James A. Williams, Lincoln, NE (US)

(21) Appl. No.: 19/567,984

(22) Filed: Mar. 16, 2026

Related U.S. Application Data
(62) Division of application No. 17/026,101, filed on Sep. 18, 2020, now Pat. No. 12,600,984.
Application No. 17/026,101 is a continuation of application No. PCT/US2019/023209, filed on Mar. 20, 2019.
(60) Provisional application No. 62/645,892, filed on Mar. 21, 2018.

(51) Int. Cl. C12N 15/85; C12N 15/70; C12N 15/86

(52) U.S. Cl. CPC C12N 15/85; C12N 15/70; C12N 15/86; C12N 2740/15043; C12N 2740/15052; C12N 2750/14143; C12N 2750/14152; C12N 2800/90; C12N 2820/55

(57) ABSTRACT

A method for improving the replication of a covalently closed circular plasmid is provided. The method includes providing a covalently closed circular plasmid having a Pol I-dependent origin of replication, and an insert including a structured DNA sequence selected from the group consisting of inverted repeat sequence, direct repeat sequence, homopolymeric repeat sequence, eukaryotic origin of replication or eukaryotic promoter enhancer sequence, wherein the structured DNA sequence is located at a distance of less than 1000 bp from the Pol I-dependent origin of replication in the direction of replication. The method also includes modifying the covalently closed circular recombinant molecule such that the Pol I-dependent origin of replication is replaced with a Pol III-dependent origin of replication, whereby the resultant Pol III-dependent origin of replication covalently closed circular plasmid has improved replication. An antibiotic marker free covalently closed circular recombinant DNA molecule is also provided.

### CROSS REFERENCE TO RELATED APPLICATIONS

This application is a divisional of U.S. patent application Ser. No. 17/026,101 filed Sep. 18, 2020, which is a continuation of International Patent Application No. PCT/US19/23209, filed Mar. 20, 2019, which claims priority to U.S. Provisional Patent Application Ser. No. 62/645,892, filed Mar. 21, 2018, the disclosures of which are incorporated herein by reference.

### STATEMENT REGARDING FEDERALLY SPONSORED RESEARCH OR DEVELOPMENT

Not applicable.

### REFERENCE TO SEQUENCE LISTING

A Sequence Listing submitted as an XML file via patent Center is hereby incorporated by reference. The name of the XML file for the Sequence Listing is “19567984.0818877_Sequence_Listing.xml”, the date of the creation of the XML file is Apr. 17, 2026, and the size of the XML file is 37,000 bytes.

### FIELD OF THE INVENTION

The present invention relates to recombinant DNA molecules, i.e., vectors, useful for viral and non-viral gene therapy, viral and non-viral cell therapy, and more particularly, for improving viral and non-viral vector manufacturing yield and quality, reducing transfection associated toxicity, improving transposition from non-viral transposon vectors, improving packaging titers from viral vectors, improving expression of viral and non-viral vector encoded genes, and for eliminating viral vector and non-viral vector mediated antibiotic selection marker gene transfer.

Such recombinant DNA molecules are useful in biotechnology, ex vivo gene therapy, transgenic organisms, gene therapy, therapeutic vaccination, agriculture and DNA vaccines.

### BACKGROUND OF THE INVENTION

E. coli plasmids have long been an important source of recombinant DNA molecules used by researchers and by industry. Today, plasmid DNA is becoming increasingly important as the next generation of biotechnology products (e.g. gene medicines and DNA vaccines) make their way into clinical trials, and eventually into the pharmaceutical marketplace. Plasmid DNA vaccines may find application as preventive vaccines for viral, bacterial, or parasitic diseases; immunizing agents for the preparation of hyper immune globulin products; therapeutic vaccines for infectious diseases; or as cancer vaccines. Plasmids are also utilized in gene therapy or gene replacement applications, wherein the desired gene product is expressed from the plasmid after administration to a patient. Plasmids are also utilized in non-viral transposon vectors for gene therapy or gene replacement applications, wherein the desired gene product is expressed from the genome after transposition from the plasmid and genome integration. Plasmids are also utilized in viral vectors for gene therapy or gene replacement applications, wherein the desired gene product is packaged in a transducing virus particle after transfection of a production cell line, and is then expressed from the virus in a target cell after viral transduction.

Non-viral and viral vector plasmids typically contain a pMB1, ColE1 or pBR322 derived replication origin. Common high copy number derivatives have mutations affecting copy number regulation, such as ROP (Repressor of primer gene) deletion, with a second site mutation that increases copy number (e.g. pMB1 pUC G to A point mutation, or ColE1 pMM1). Higher temperature (42° C.) can be employed to induce selective plasmid amplification with pUC and pMM1 replication origins.

Carnes, A E and Williams, J A, 2011 U.S. Pat. No. 7,943,377 describe methods for fed-batch fermentation, in which plasmid-containing E. coli cells were grown at a reduced temperature during part of the fed-batch phase, during which growth rate was restricted, followed by a temperature up-shift and continued growth at elevated temperature in order to accumulate plasmid; the temperature shift at restricted growth rate improved plasmid yield and purity. Other fermentation processes for plasmid production are described in Carnes A. E. 2005 BioProcess Intl 3:36-44, which is incorporated herein by reference in its entirety.

The art teaches that one of the limitations of application of plasmid therapies and plasmid vaccines is regulatory agency (e.g. Food and Drug Administration, European Medicines Agency) safety concerns regarding: 1) plasmid transfer and replication in endogenous bacterial flora, or 2) plasmid encoded selection marker expression in human cells, or endogenous bacterial flora. Additionally, regulatory agency guidance's recommend removal of all non-essential sequences in a vector. Plasmids containing a pMB1, ColE1 or pBR322 derived replication origin can replicate promiscuously in E. coli hosts. This presents a safety concern that a plasmid therapeutic gene or antigen will be transferred to, and replicated within, a patient's endogenous flora. Ideally, a therapeutic or vaccine plasmid would be replication incompetent in endogenous E. coli strains. This requires replacement of the pMB1, ColE1 or pBR322 derived replication origin with a conditional replication origin that requires a specialized cell line for propagation. As well, regulatory agencies, such as the EMEA and FDA, are concerned with utilization of antibiotic resistance or alternative protein markers in gene therapy and gene vaccine vectors, due to concerns that the gene (antibiotic resistance marker or protein marker) may be expressed in a patient's cells. Ideally, plasmid therapies and plasmid vaccines would: 1) be replication incompetent in endogenous E. coli strains, 2) not encode a protein-based selection marker and 3) be minimalized to eliminate all non-essential sequences.

The art further teaches that one of the limitations of application of plasmid vectors is that transgene expression duration from plasmid vectors is reduced due to promoter inactivation mediated by the bacterial region (i.e. the region encoding the bacterial replication origin and selectable marker) of the vector (Chen Z Y, He C Y, Meuse L, Kay M A. 2004. Gene Ther 11:856-864; Suzuki M, Kasai K, Saeki Y. 2006. J Virol 80:3293-3300). This results in short duration transgene expression. A strategy to improve transgene expression duration is to remove the bacterial region of the plasmid. For example, minicircle vectors have been developed which do not contain a bacterial region. Removal of the bacterial region in minicircle vectors improved transgene expression duration (Chen et al., Supra, 2004). In minicircle vectors, the eukaryotic region polyadenylation signal is covalently linked to the eukaryotic region promoter through a short spacer typically less than 200 bp comprised of the recombined attachment sites. This linkage (spacer region) can tolerate a much longer spacer sequence since while long spacers ≥1 kb in length resulted in transgene expression silencing in vivo, shorter spacers ≤500 bp exhibited similar transgene expression patterns to conventional minicircle DNA vectors (Lu J, Zhang F, Xu S, Fire A Z, Kay M A. 2012. Mol Ther. 20:2111-9).

Williams, 2014. DNA plasmids with improved expression. World Patent Application WO2014035457 disclose minimalized Nanoplasmid™ vectors utilize RNA-OUT antibiotic-free selection and replace the large 1000 bp pUC replication origin with a novel, 300 bp, R6K origin. Reduction of the spacer region linking the 5′ and 3′ ends of the transgene expression cassette to <500 bp with R6K origin-RNA-OUT backbones improved expression duration to that of conventional minicircle DNA vectors as expected from the teachings of Lu et al., Supra, 2012.

The 1.1 kb pFAR4 vector pUC-origin tRNA antibiotic free selection spacer has improved expression duration compared to a 2.2 kb pUC origin-kanR antibiotic selection marker spacer region (Quiviger, M, Arfi A, Mansard D, Delacotte L, Pastor M, Scherman D, Marie C. 2014. Gene Therapy 21:1001-1007). This teaches that improved expression duration can be obtained with some bacterial regions up to 1.1 kb.

Expression level improvement compared to plasmid vectors is also observed with some spacer regions 1.1 kb. For example, pVAX1 derivatives with the 2 kb bacterial backbone reduced to 1.2, 1.1 or 0.7 kb show ≥2-fold improved expression compared to the parent pVAX1 vector (Table 1). NTC8685 derivatives with the 1.5 kb bacterial backbone reduced to 0.9 kb, 466 bp or 281 bp (Nanoplasmid™ vectors) show 2-fold improved expression compared to the parent NTC8685 vector (Table 2).

This teaches that improved expression level can be obtained with short bacterial regions up to 1.2 kb.

TABLE 1
pVAX1 mammalian expression vector spacer region (SR) derivatives expression level

| pVAX1 Vector | Bacterial | | Selection | | In Vitro | In vivo |
|---|---|---|---|---|---|---|
| derivatives | Backbone (bp) | | Marker | Selection | Expression | Expression |
| pVAX1 | 2 | kb | KanR | Kan | 1X (baseline) | 1X (baseline) |
| pVAX1-A2^{a} | 1.2 | kb | RNA-OUT | Sucrose | 4x | ND |
| pVAX1/MINI^{b} | 0.7 | kb | RNAI | murA | 2x | ND |
| | | | (pUC origin) | translation | | |
| pFAR4^{c} | 1.1 | kb | tRNA | thyA amber | ND | >5x ID EP |
| | | | (amber) | mutation | | |
| | | | | suppression | | |
| Minicircle^{d} | 50 | bp | NA, | NA, | 1.4-1.6x | 1.3x IM |
| | | | Minicircle | Minicircle | (equimolar dose) | (equimolar dose) |

^{a}Carnes et al., (2010) J. Gene Medicine 12: 818-31
^{b}Ribeiro et al., (2011) Cell Reprogram 14: 130
^{c}Marie et al., (2010) J. Gene Medicine 12: 323-32
^{d}Wang et al., (2014) J Virology 88: 1924-34

TABLE 2
NTC8685 mammalian expression vector spacer region (SR) derivatives expression level

| NTC8685 Vector | Replication | Bacterial | | Selection | In vitro | In vivo |
|---|---|---|---|---|---|---|
| derivatives^{a} | origin | backbone (bp) | | Marker | Expression (A549) | Expression (ID + EP) |
| NTC8685 | pUC origin | 1.5 | kb | RNA-OUT | 1X (baseline) | 1X (baseline) |
| NTC8385-Min | Minimalized | 0.9 | kb | RNA-OUT | 2.1x | 1.4-1.9x |
| | pUC origin | | | | | |
| NTC9385R | R6K origin | 466 | bp | RNA-OUT | 2.5x | 5.3-6.3x |
| Nanoplasmid ™ | | | | | | |
| NTC9385C | ColE2 origin | 281 | bp | RNA-OUT | 1.1x | 2.7-3.1x |
| Nanoplasmid ™ | | | | | | |

^{a}Williams, Supra, 2014

Various investigators have identified that minicirce vectors are superior to plasmid vectors for production of AAV vectors (improved transducing unit titers—Table 3) and transposon vectors (increased transposition—Table 3). The improved performance due to improved expression duration with short backbone minicircle vectors should also be observed with short bacterial backbone plasmid vectors up to 1.1 kb.

TABLE 3
Minicircle applications with various viral and non-viral vector platforms

| | pUC Plasmid | Minicircle | | Result- |
|---|---|---|---|---|
| | (>1.5 kb pUC | (MC) retrofit | | antibiotic |
| Vector | origin-antibiotic | (<400 bp | Result- | marker gene |
| platform | marker spacer region) | spacer region) | performance | transfer |
| self- | pAAV- | MC.AAVscGFP | Up to 30-fold improved | Plasmid backbone |
| complementary | scGFP | | Transducing units^{a} | antibiotic resistance |
| (sc) AAV | | | | marker packaged in up |
| | | | | to 26.1% viral particles^{a} |
| | | | | potential for genome |
| | | | | integration in transduced |
| | | | | cells |
| single-stranded | AAV2- | MC.AAV- | Up to 3-fold improved | Plasmid backbone |
| (ss) AAV | ssGFP | ssGFP | Transducing units^{a} | antibiotic resistance |
| | | | | marker packaged in up |
| | | | | to 2.9%^{a} or |
| | | | | 3%^{b} of viral particles |
| | | | | potential for genome |
| | | | | integration in transduced |
| | | | | cells |
| Sleeping | SB puroR | SB puroR | 2-fold increased | Plasmid backbone |
| Beauty | reporter | reporter MC | transposition rate | antibiotic resistance |
| Transposon | plasmid | | into established | potential for genome |
| | | | cell lines^{c} | integration in T-cells |
| | SB CD19- | MC: SB CD19 | 4-fold increased yield of | |
| | CAR | CAR | CD19-CAR T-cells; reduced | |
| | | | transfection associated | |
| | | | toxicity^{d} | |
| | SB | MC: SB | 6-7-fold increased | |
| | | (218 bp MC | transposition into | |
| | | Backbone) | Human Hematopoietic | |
| | | | Stem cells; reduced | |
| | | | transfection associated | |
| | | | toxicity^{e} | |

^{a}Schnödt et al., (2016) Mol Ther - Nucleic Acids 5 e355. This citation, along with Chadeuf et al., (2005) Molecular Therapy 12: 744-53 and Gray, 2017. WO2017066579 also reports that AAV helper plasmid antibiotic resistance markers are packaged into viral particles, demonstrating need to remove antibiotic markers from AAV helper plasmids as well as the AAV vector. Interestingly, no improvement in viral titer compared to plasmid is observed with linear Doggybone ™ DNA single-stranded (ss) AAV vector retrofits (Karbownickez et al., 2017. Cell and Gene Therapy Insights 731-8). This suggests that short backbone minicircle vector improvement compared to >1.5 kb pUC-antibiotic marker plasmid backbone vectors require a short backbone circular vector rather than a linear vector such as Doggybone ™ vectors.
^{b}Gray, Supra, 2017 describes adding a large spacer region to move selection marker away from ITRs. Resultant vector, pAAV CMV GFP is 11 kb compared to 5-6 kb for standard plasmid AAV vector so will have reduced transducing units compared to parental
^{c}Sharma et al., (2013) Molecular Therapy Nucleic Acids 2: e74
^{d}Nucleofection transfection: Monjezi et al., (2017) Leukemia 31: 186-94
^{e}Nucleofection transfection: Holstein et al., (2018) Molecular Therapy epub Jan. 17, 2018

Indeed 2-fold improved sleeping beauty transposition into human cells was reported with the 1.1 kb bacterial backbone pFAR4 SB transposon vector/SB100× transposase vector combination compared to a 2.8 kb bacterial backbone pT2 plasmid SB transposon vector/SB100× transposase vector combination (Pastor, M, Johnen S, Harmening N, Quiviger M, Pailloux J, Kropp, M, Walter P, Ivics Z, Izsvak Z, Thumann G, Scherman D. 2018 Molecular Therapy 11: 57-67).

However, viral vectors such as AAV, Lentiviral and Retroviral vectors, and transposon vectors contained structured DNA sequences at their termini. For example, Sleeping Beauty transposon vectors contain flanking IR/DR sequences, AAV vectors contain flanking ITRs, and Lentiviral and Retroviral vectors contain flanking LTRs.

The close proximity of the pUC origin to a structured DNA sequence results in aberrant replication termination, resulting in replication intermediates which unacceptably reduce plasmid quality (Levy J. 2004. U.S. Pat. No. 6,709,844). Levy teaches that replication intermediates form when any high copy replication origin is <1 kb from a structured DNA sequence such as an enhancer, LTR or IRES, but not when the high copy replication origin is >1.5 kb away. Since the pUC origin itself is 1 kb, there is no configuration to make a <1.1 kb bacterial region AAV, Lentiviral, Retroviral or transposon vector containing the pUC origin which is not predicted to produce replication intermediates.

Lu J, Williams J A, Luke J, Zhang F, Chu K, and Kay M A. 2017. Human Gene Therapy 28:125-34 disclose antibiotic free Mini-Intronic Plasmid (MIP) AAV vectors and suggest that MIP intron AAV vectors could have the vector backbone removed to create a short backbone AAV vector. Attempts to create a minicircle like 6 or 10 bp spacer region in Mini-Intronic Plasmid AAV vectors were toxic (see Table 7, footnote e) presumably due to creation of a long palindrome by such close juxtaposition of the AAV ITRs. While MIP vectors with longer spacer regions <1 kb can be made, a drawback of the MIP intron strategy is that it requires cloning of a replication and selection encoding intron into the eukaryotic region, which is not possible or desired with many vectors.

A drawback of the minicircle strategy to create short bacterial region AAV, Lentiviral, Retroviral or transposon vectors, is that methods to manufacture minicircle vectors are expensive and not easily scalable. For minicircle vectors, E. coli-based manufacturing systems have been developed in which, after plasmid production, the bacterial region and the eukaryotic region are separated and circularized into a minicircle (eukaryotic region) and a bacterial region circle via the action of phage recombinases on recognition sequences in the plasmid. In some methods, a restriction enzyme is then utilized to digest the bacterial region circle at a unique site to eliminate this difficult to remove contaminant. These production procedures are very inefficient. For example, optimal manufacture of minicircle vectors yields only 5 mg of minicircle per liter culture (Kay M A, He C Y, Chen Z Y. 2010. Nat Biotechnol 28:1287-1289).

Methods for high yield manufacture of pFAR vectors have not been reported; this system utilizes a plasmid bome suppressor tRNA gene to complement a TAG amber nonsense mutation of the thyA gene to complement thymidine auxotrophy and allow cell growth on minimal media (Marie et al., Supra, 2010).

A solution is needed to develop AAV, Lentiviral, Retroviral or transposon vector that contain short spacer regions preferably less than 1000 bp that can be efficiently manufactured without replication intermediates or poor production. These vectors should not encode a protein-based selection marker and should be minimalized to eliminate all non-essential sequences.

### SUMMARY OF THE INVENTION

The present invention relates to vectors useful for viral and non-viral gene therapy, viral and non-viral cell therapy, and more particularly, for improving viral and non-viral vector manufacturing yield and quality, for reducing transfection associated toxicity, for improving transposition from non-viral transposon vectors, for improving packaging titers from viral vectors, for improving expression of viral and non-viral vector encoded transgenes, and for eliminating antibiotic resistance marker gene transfer by viral and non-viral vectors.

Improved vector methods and compositions that utilize a Pol III-dependent origin of replication to replicate structured DNA sequences are disclosed.

Improved vector methods and compositions that utilize a Pol III-dependent origin of replication to replicate inverted repeat DNA sequences are disclosed.

Improved vector methods and compositions that utilize a Pol III-dependent origin of replication to replicate direct repeat DNA sequences are disclosed.

Improved vector methods and compositions that utilize a Pol III-dependent origin of replication to replicate homopolymeric repeat DNA sequences are disclosed.

Improved vector methods and compositions that utilize a Pol III-dependent origin of replication to replicate enhancer structured DNA sequences are disclosed.

Improved vector methods and compositions that utilize a Pol III-dependent origin of replication to replicate polyA repeat DNA sequences are disclosed.

Improved vector methods and compositions that utilize a Pol III-dependent origin of replication to replicate SV40 origin of replication DNA sequences are disclosed.

Improved vector methods and compositions that utilize a Pol III-dependent origin of replication to replicate Lentiviral LTR DNA sequences are disclosed.

Improved vector methods and compositions that utilize a Pol III-dependent origin of replication to replicate Retroviral LTR DNA sequences are disclosed.

Improved vector methods and compositions that utilize a Pol III-dependent origin of replication to replicate viral LTR DNA sequences are disclosed.

Improved vector methods and compositions that utilize a Pol III-dependent origin of replication to replicate AAV ITR DNA sequences are disclosed.

Improved vector methods and compositions that utilize a Pol III-dependent origin of replication to replicate transposon IR/DR DNA sequences are disclosed.

Improved vector methods and compositions that utilize a Pol III-dependent origin of replication to replicate Sleeping Beauty IR/DR DNA sequences are disclosed.

Improved vector methods and compositions that utilize a Pol III-dependent origin of replication to replicate PiggyBac ITR DNA sequences are disclosed.

Improved vector methods and compositions that utilize a Pol III-dependent origin of replication to replicate CMV enhancer DNA sequences are disclosed.

Improved vector methods and compositions that utilize a Pol III-dependent origin of replication to replicate direct SV40 enhancer DNA sequences are disclosed.

Improved viral vector methods and compositions that utilize a Pol III-dependent origin of replication are disclosed.

Improved Lentiviral vector, Lentiviral envelope vector and Lentiviral packaging vector methods and compositions that utilize a Pol III-dependent origin of replication are disclosed.

Improved Retroviral vector, Retroviral envelope vector and Retroviral packaging vector methods and compositions that utilize a Pol III-dependent origin of replication are disclosed.

Improved AAV vector and AAV helper vector methods and compositions that utilize a Pol III-dependent origin of replication are disclosed.

Improved Adenoviral vector methods and compositions that utilize a Pol III-dependent origin of replication are disclosed.

Improved non-viral transposon and transposase vector methods and compositions that utilize a Pol III-dependent origin of replication are disclosed.

Improved non-viral Sleeping Beauty transposon and transposase vector methods and compositions that utilize a Pol III-dependent origin of replication are disclosed.

Improved non-viral PiggyBac transposon and transposase vector methods and compositions that utilize a Pol III-dependent origin of replication are disclosed.

Improved non-viral Tol2 transposon and transposase vector methods and compositions that utilize a Pol III-dependent origin of replication are disclosed.

Improved non-viral polyA containing mRNA vector methods and compositions that utilize a Pol III-dependent origin of replication are disclosed Improved viral vector methods and compositions with improved viral transducing unit production that utilize a Pol III-dependent origin of replication are disclosed.

Improved Lentiviral vector methods and compositions with improved viral transducing unit production that utilize a Pol III-dependent origin of replication are disclosed.

Improved Retroviral vector methods and compositions with improved viral transducing unit production that utilize a Pol III-dependent origin of replication are disclosed.

Improved AAV vector and AAV helper vector methods and compositions with improved viral transducing unit production that utilize a Pol III-dependent origin of replication are disclosed.

Improved non-viral transposon and transposase vector methods and compositions with improved transposition that utilize a Pol III-dependent origin of replication are disclosed.

Improved non-viral Sleeping Beauty transposon and transposase vector methods and compositions with improved transposition that utilize a Pol III-dependent origin of replication are disclosed.

Improved non-viral PiggyBac transposon and transposase vector methods and compositions with improved transposition that utilize a Pol III-dependent origin of replication are disclosed.

Improved non-viral Tol2 transposon and transposase vector methods and compositions with improved transposition that utilize a Pol III-dependent origin of replication are disclosed.

Improved viral vector methods and compositions with improved expression that utilize a Pol III-dependent origin of replication are disclosed.

Improved Lentiviral vector methods and compositions with improved expression that utilize a Pol III-dependent origin of replication are disclosed.

Improved Retroviral vector methods and compositions with improved expression that utilize a Pol III-dependent origin of replication are disclosed.

Improved AAV vector and AAV helper vector methods and compositions with improved expression that utilize a Pol III-dependent origin of replication are disclosed.

Improved non-viral transposon and transposase vector methods and compositions with improved expression that utilize a Pol III-dependent origin of replication are disclosed.

Improved non-viral Sleeping Beauty transposon and transposase vector methods and compositions with improved expression that utilize a Pol III-dependent origin of replication are disclosed.

Improved non-viral PiggyBac transposon and transposase vector methods and compositions with improved expression that utilize a Pol III-dependent origin of replication are disclosed.

Improved non-viral Tol2 transposon and transposase vector methods and compositions with improved expression that utilize a Pol III-dependent origin of replication are disclosed.

Improved viral vector methods and compositions with no antibiotic resistance marker gene transfer risk that utilize a Pol III-dependent origin of replication are disclosed.

Improved Lentiviral vector, Lentiviral envelope vector and Lentiviral packaging vector methods and compositions with no antibiotic resistance marker gene transfer risk that utilize a Pol III-dependent origin of replication are disclosed.

Improved Retroviral vector, Retroviral envelope vector and Retroviral packing vector methods and compositions with no antibiotic resistance marker gene transfer risk that utilize a Pol III-dependent origin of replication are disclosed.

Improved AAV vector and AAV helper vector methods and compositions with no antibiotic resistance marker gene transfer risk that utilize a Pol III-dependent origin of replication are disclosed.

Improved non-viral transposon and transposase vector methods and compositions with no antibiotic resistance marker gene transfer risk that utilize a Pol III-dependent origin of replication are disclosed.

Improved non-viral Sleeping Beauty transposon and transposase vector methods and compositions with no antibiotic resistance marker gene transfer risk that utilize a Pol III-dependent origin of replication are disclosed.

Improved non-viral PiggyBac transposon and transposase vector methods and compositions with no antibiotic resistance marker gene transfer risk that utilize a Pol III-dependent origin of replication are disclosed.

Improved non-viral Tol2 transposon and transposase vector methods and compositions with no antibiotic resistance marker gene transfer risk that utilize a Pol III-dependent origin of replication are disclosed.

Improved viral vector methods and compositions with reduced transfection associated toxicity that utilize a Pol III-dependent origin of replication are disclosed.

Improved Lentiviral vector methods and compositions with reduced transfection associated toxicity that utilize a Pol III-dependent origin of replication are disclosed.

Improved Retroviral vector methods and compositions with reduced transfection associated toxicity that utilize a Pol III-dependent origin of replication are disclosed.

Improved AAV vector and AAV helper vector methods and compositions with reduced transfection associated toxicity that utilize a Pol III-dependent origin of replication are disclosed.

Improved non-viral transposon and transposase vector methods and compositions with reduced transfection associated toxicity that utilize a Pol III-dependent origin of replication are disclosed.

Improved non-viral Sleeping Beauty transposon and transposase vector methods and compositions with reduced transfection associated toxicity that utilize a Pol III-dependent origin of replication are disclosed.

Improved non-viral PiggyBac transposon and transposase vector methods and compositions with reduced transfection associated toxicity that utilize a Pol III-dependent origin of replication are disclosed.

Improved non-viral Tol2 transposon and transposase vector methods and compositions with reduced transfection associated toxicity that utilize a Pol III-dependent origin of replication are disclosed.

Each of the above improvements and the improvements described below is, for example, relative to what is achieved in similar or identical circumstances, but with a different plasmid that does not include a Pol III-dependent original of replication.
One object of the invention is to provide improved viral and non-viral vector manufacturing yield.

Another object of the invention is to provide improved viral and non-viral vector manufacturing quality.

Another object of the invention is to provide viral vectors with improved packaging titers.

Another object of the invention is to provide non-viral transposon vectors with improved transposition.

Another object of the invention is to provide viral and non-viral vectors with improved expression of encoded transgenes.

Another object of the invention is to provide viral and non-viral vectors that eliminate antibiotic resistance marker gene transfer.

Another object of the invention is to provide viral and non-viral vectors with reduced transfection associated toxicity.

In one embodiment, the present technology provides a method for improving the replication of a covalently closed circular plasmid comprising the following steps: a) providing a covalently closed circular plasmid comprising: i) a Pol I-dependent origin of replication, and ii) an insert comprising a structured DNA sequence selected from the group consisting of inverted repeat sequence, direct repeat sequence, homopolymeric repeat sequence, eukaryotic origin of replication and eukaryotic promoter enhancer sequence, wherein the structured DNA sequence is located at a distance of less than 1000 bp from the Pol I-dependent origin of replication in the direction of replication; b) modifying the covalently closed circular recombinant molecule of a) such that the Pol I-dependent origin of replication is replaced with a Pol III-dependent origin of replication whereby the resultant Pol III-dependent origin of replication covalently closed circular plasmid has improved replication. In a further embodiment said Pol I-dependent origin of replication is selected from the group consisting of: pUC origin, pMB1 origin, and ColE1 origin. In a further embodiment said Pol III-dependent origin of replication is an R6K gamma replication origin. In a further embodiment said Pol III-dependent origin of replication is an R6K gamma replication origin with at least 95% sequence identity to a sequence selected from the group consisting of SEQ ID NO: 1, SEQ ID NO: 2, SEQ ID NO: 3, SEQ ID NO: 4 and SEQ ID NO: 18. In a further embodiment said structured DNA sequence is selected from the group consisting of polyA repeat, SV40 origin of replication, viral LTR, Lentiviral LTR, Retroviral LTR, transposon IR/DR repeat, Sleeping Beauty transposon IR/DR repeat, AAV ITR, CMV enhancer, and SV40 enhancer. In a further embodiment said improved replication is selected from the group consisting of reduced production of replication intermediates and increased plasmid copy number.

In another embodiment, the present technology provides a method for improving the replication of a covalently closed circular plasmid comprising the following steps: a) providing a covalently closed circular plasmid comprising: i) a bacterial replication-selection region comprising a Pol I-dependent origin of replication and an antibiotic selectable marker, and ii) an insert comprising a structured DNA sequence selected from the group consisting of inverted repeat sequence, direct repeat sequence, homopolymeric repeat sequence, eukaryotic origin of replication and eukaryotic promoter enhancer sequence, wherein the structured DNA sequence is located at a distance of less than 1000 bp from the Pol I-dependent origin of replication in the direction of replication; b) modifying the covalently closed circular recombinant molecule of a) such that the antibiotic selectable marker is replaced with an RNA selectable marker and the Pol I-dependent origin of replication is replaced with a Pol III-dependent origin of replication, whereby the resultant Pol III-dependent origin of replication covalently closed circular plasmid has improved replication. In a further embodiment said Pol I-dependent origin of replication is selected from the group consisting of: pUC origin, pMB1 origin, and ColE1 origin. In a further embodiment said Pol III-dependent origin of replication is an R6K gamma replication origin. In a further embodiment said Pol III-dependent origin of replication is an R6K gamma replication origin with at least 95% sequence identity to a sequence selected from the group consisting of SEQ ID NO: 1, SEQ ID NO: 2, SEQ ID NO: 3, SEQ ID NO: 4 and SEQ ID NO: 18. In a further embodiment said RNA selectable marker is an RNA-IN regulating RNA-OUT functional variant with at least 95% sequence identity to a sequence selected from the group consisting of SEQ ID NO: 5, and SEQ ID NO: 7. In a further embodiment said RNA selectable marker is an RNA-OUT RNA selectable marker that encodes an RNA-IN regulating RNA-OUT RNA with at least 95% sequence identity to SEQ ID NO: 6. In a further embodiment said bacterial replication-selection region comprising a Pol I-dependent origin of replication and an antibiotic selectable marker is replaced with a Pol III-dependent R6K origin-RNA-OUT RNA selectable marker bacterial replication-selection region with at least 95% sequence identity to a sequence selected from the group consisting of SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17. In a further embodiment said structured DNA sequence is selected from the group consisting of polyA repeat, SV40 origin of replication, viral LTR, Lentiviral LTR, Retroviral LTR, transposon IR/DR repeat, Sleeping Beauty transposon IR/DR repeat, AAV ITR, CMV enhancer, and SV40 enhancer. In a further embodiment said improved replication is selected from the group consisting of reduced production of replication intermediates, and increased plasmid copy number.

In one embodiment, the current technology provides an antibiotic marker free covalently closed circular recombinant DNA molecule comprising: a) an antibiotic marker free insert comprising a structured DNA sequence selected from the group consisting of inverted repeat sequence, direct repeat sequence, homopolymeric repeat sequence, eukaryotic origin of replication, and eukaryotic promoter enhancer sequence; b) a Pol III-dependent origin of replication comprising an R6K gamma replication origin with at least 95% sequence identity to a sequence selected from the group consisting of SEQ ID NO: 1, SEQ ID NO: 2, SEQ ID NO: 3, SEQ ID NO: 4 and SEQ ID NO: 18; and c) an RNA-OUT RNA selectable marker comprising an RNA-IN regulating RNA-OUT functional variant with at least 95% sequence identity to a sequence selected from the group consisting of SEQ ID NO: 5, and SEQ ID NO: 7. In a further embodiment said R6K gamma replication origin and said RNA-OUT RNA selectable marker comprise a R6K origin-RNA-OUT RNA selectable marker bacterial replication-selection region with at least 95% sequence identity to a sequence selected from the group consisting of SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17. In a further embodiment said structured DNA sequence is selected from the group consisting of polyA repeat, SV40 origin of replication, viral LTR, Lentiviral LTR, Retroviral LTR, transposon IR/DR repeat, Sleeping Beauty transposon IR/DR repeat, AAV ITR, CMV enhancer, and SV40 enhancer. In a further embodiment said recombinant DNA molecule is selected from the group consisting of viral vector, Lentiviral vector, Retroviral vector, AAV vector, Ad vector, non-viral transposon vector, Sleeping Beauty transposon vector, PiggyBac transposon vector, Tol2 transposon vector, and polyA containing mRNA vector.

In one embodiment, the present technology provides a method for improving AAV vector viral transducing unit production from a covalently closed circular plasmid comprising the following steps: a) providing a covalently closed circular plasmid comprising: i) a 1 kb or larger bacterial replication-selection region comprising a Pol I-dependent origin of replication and an antibiotic selectable marker, and ii) an insert comprising a eukaryotic region selected from the group consisting of AAV vector, AAV rep cap vector, Ad helper vector, and Ad helper rep cap vector; b) modifying the covalently closed circular recombinant molecule of a) such that the 1 kb or larger bacterial replication-selection region comprising a Pol I-dependent origin of replication and an antibiotic selectable marker is replaced with an less than 1 kb Pol Ill-dependent R6K origin-RNA-OUT RNA selectable marker bacterial replication-selection region, whereby the resultant Pol Ill-dependent origin of replication covalently closed circular plasmid has improved AAV viral transducing unit production when transfected into mammalian cells. In a further embodiment said Pol I-dependent origin of replication is selected from the group consisting of pUC origin, pMB1 origin, and ColE1 origin. In a further embodiment said Pol Ill-dependent R6K origin of replication is an R6K gamma replication origin with at least 95% sequence identity to a sequence selected from the group consisting of SEQ ID NO: 1, SEQ ID NO: 2, SEQ ID NO: 3, SEQ ID NO: 4 and SEQ ID NO: 18. In a further embodiment said RNA-OUT RNA selectable marker is an RNA-IN regulating RNA-OUT functional variant with at least 95% sequence identity to a sequence selected from the group consisting of SEQ ID NO: 5, and SEQ ID NO: 7. In a further embodiment said RNA-OUT RNA selectable marker is an RNA-OUT RNA selectable marker that encodes an RNA-IN regulating RNA-OUT RNA with at least 95% sequence identity to SEQ ID NO: 6. In a further embodiment said less than 1 kb Pol Ill-dependent R6K origin-RNA-OUT RNA selectable marker bacterial replication-selection region has at least 95% sequence identity to a sequence selected from the group consisting of SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

In one embodiment, the current technology provides a method for improving Retroviral or Lentiviral vector viral transducing unit production from a covalently closed circular plasmid comprising the following steps: a) providing a covalently closed circular plasmid comprising: i) a 1 kb or larger bacterial replication-selection region comprising a Pol I-dependent origin of replication and an antibiotic selectable marker, and ii) an insert comprising a eukaryotic region selected from the group consisting of Retroviral vector, Lentiviral vector, Retroviral envelope vector, Lentiviral envelope vector, Retroviral packaging vector and Lentiviral packaging vector; and b) modifying the covalently closed circular recombinant molecule of a) such that the 1 kb or larger bacterial replication-selection region comprising a Pol I-dependent origin of replication and an antibiotic selectable marker is replaced with an less than 1 kb Pol III-dependent R6K origin-RNA-OUT RNA selectable marker bacterial replication-selection region, whereby the resultant Pol III-dependent origin of replication covalently closed circular plasmid has improved viral transducing unit production when transfected into mammalian cells. In a further embodiment said Pol I-dependent origin of replication is selected from the group consisting of: pUC origin, pMB1 origin, and ColE1 origin. In a further embodiment said Pol III-dependent R6K origin of replication is an R6K gamma replication origin with at least 95% sequence identity to a sequence selected from the group consisting of SEQ ID NO: 1, SEQ ID NO: 2, SEQ ID NO: 3, SEQ ID NO: 4 and SEQ ID NO: 18. In a further embodiment said RNA-OUT RNA selectable marker is an RNA-IN regulating RNA-OUT functional variant with at least 95% sequence identity to a sequence selected from the group consisting of SEQ ID NO: 5, and SEQ ID NO: 7. In a further embodiment said RNA-OUT RNA selectable marker is an RNA-OUT RNA selectable marker that encodes an RNA-IN regulating RNA-OUT RNA with at least 95% sequence identity to SEQ ID NO: 6. In a further embodiment said less than 1 kb Pol III-dependent R6K origin-RNA-OUT RNA selectable marker bacterial replication-selection region has at least 95% sequence identity to a sequence selected from the group consisting of SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

In one embodiment, the present invention contemplates a method for improving transposition from a covalently closed circular non-viral transposon plasmid comprising the following steps: a) providing a covalently closed circular plasmid comprising i) a 1 kb or larger bacterial replication-selection region comprising a Pol I-dependent origin of replication and an antibiotic selectable marker, and ii) an insert comprising a non-viral eukaryotic region selected from the group consisting of transposon vector, Sleeping Beauty transposon vector, Sleeping Beauty transposase vector, PiggyBac transposon vector, PiggyBac transposase vector, Tol2 transposon vector, Tol2 transposase vector; b) modifying the covalently closed circular recombinant molecule of (a) such that the 1 kb or larger bacterial replication-selection region comprising a Pol I-dependent origin of replication and an antibiotic selectable marker is replaced with an less than 1 kb Pol III-dependent R6K origin-RNA-OUT RNA selectable marker bacterial replication-selection region, whereby the resultant Pol III-dependent origin of replication covalently closed circular plasmid has improved transposition when transfected into mammalian cells. In a further embodiment said Pol I-dependent origin of replication is selected from the group consisting of: pUC origin, pMB1 origin, or ColE1 origin. In a further embodiment said Pol III-dependent R6K origin of replication is an R6K gamma replication origin with at least 95% sequence identity to a sequence selected from the group consisting of SEQ ID NO: 1, SEQ ID NO: 2, SEQ ID NO: 3, SEQ ID NO: 4 and SEQ ID NO: 18. In a further embodiment said RNA-OUT RNA selectable marker is an RNA-IN regulating RNA-OUT functional variant with at least 95% sequence identity to a sequence selected from the group consisting of SEQ ID NO: 5, SEQ ID NO: 7. In a further embodiment said RNA-OUT RNA selectable marker is an RNA-OUT RNA selectable marker that encodes an RNA-IN regulating RNA-OUT RNA with at least 95% sequence identity to SEQ ID NO: 6. In a further embodiment said less than 1 kb Pol III-dependent R6K origin-RNA-OUT RNA selectable marker bacterial replication-selection region has at least 95% sequence identity to a sequence selected from the group consisting of SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17.

In one embodiment, current technology provides a method for improving expression from a covalently closed circular viral vector or non-viral transposon plasmid comprising the following steps: a) providing a covalently closed circular plasmid comprising: i) a 1 kb or larger bacterial replication-selection region comprising a Pol I-dependent origin of replication and an antibiotic selectable marker, and ii) an insert comprising a eukaryotic region selected from the group consisting of Lentiviral vector, Retroviral vector, and AAV vector or non-viral transposon vector; and b) modifying the covalently closed circular recombinant molecule of (a) such that the 1 kb or larger bacterial replication-selection region comprising a Pol I-dependent origin of replication and an antibiotic selectable marker is replaced with an less than 1 kb Pol III-dependent R6K origin-RNA-OUT RNA selectable marker bacterial replication-selection region, whereby the resultant Pol III-dependent origin of replication covalently closed circular plasmid has improved expression when transfected into mammalian cells. In a further embodiment said Pol I-dependent origin of replication is selected from the group consisting of: pUC origin, pMB1 origin, and ColE1 origin. In a further embodiment said Pol III-dependent R6K origin of replication is an R6K gamma replication origin with at least 95% sequence identity to a sequence selected from the group consisting of SEQ ID NO: 1, SEQ ID NO: 2, SEQ ID NO: 3, SEQ ID NO: 4 and SEQ ID NO: 18. In a further embodiment said RNA-OUT RNA selectable marker is an RNA-IN regulating RNA-OUT functional variant with at least 95% sequence identity to a sequence selected from the group consisting of SEQ ID NO: 5, and SEQ ID NO: 7. In a further embodiment said RNA-OUT RNA selectable marker is an RNA-OUT RNA selectable marker that encodes an RNA-IN regulating RNA-OUT RNA with at least 95% sequence identity to SEQ ID NO: 6. In a further embodiment said less than 1 kb Pol III-dependent R6K origin-RNA-OUT RNA selectable marker bacterial replication-selection region has at least 95% sequence identity to a sequence selected from the group consisting of SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

In one embodiment, the present technology provides a method for eliminating antibiotic resistant marker gene transfer from a covalently closed circular viral vector plasmid comprising the following steps: a) providing a covalently closed circular plasmid comprising: i) a 1 kb or larger bacterial replication-selection region comprising a Pol I-dependent origin of replication and an antibiotic resistance marker, and ii) an insert comprising an antibiotic resistance marker free eukaryotic region selected from the group consisting of viral vector, Lentiviral vector, Lentiviral packaging vector, Lentiviral envelope vector Retroviral vector, Retroviral envelope vector, Retroviral packaging vector, AAV vector, AAV rep cap vector, Ad helper vector, and Ad helper rep cap vector; and b) modifying the covalently closed circular recombinant molecule of a) such that the 1 kb or larger bacterial replication-selection region comprising a Pol I-dependent origin of replication and an antibiotic selectable marker is replaced with an less than 1 kb Pol III-dependent R6K origin-RNA-OUT RNA selectable marker bacterial replication-selection region, whereby the resultant Pol III-dependent origin of replication covalently closed circular plasmid has no antibiotic resistance markers that could be packaged into Lentiviral, Retroviral or AAV transducing viral particles when transfected into mammalian cells. In a further embodiment said Pol I-dependent origin of replication is selected from the group consisting of pUC origin, pMB1 origin, and ColE1 origin. In a further embodiment said Pol III-dependent R6K origin of replication is an R6K gamma replication origin with at least 95% sequence identity to a sequence selected from the group consisting of SEQ ID NO: 1, SEQ ID NO: 2, SEQ ID NO: 3, SEQ ID NO: 4 and SEQ ID NO: 18. In a further embodiment said RNA-OUT RNA selectable marker is an RNA-IN regulating RNA-OUT functional variant with at least 95% sequence identity to a sequence selected from the group consisting of SEQ ID NO: 5, and SEQ ID NO: 7. In a further embodiment said RNA-OUT RNA selectable marker is an RNA-OUT RNA selectable marker that encodes an RNA-IN regulating RNA-OUT RNA with at least 95% sequence identity to SEQ ID NO: 6. In a further embodiment said less than 1 kb Pol III-dependent R6K origin-RNA-OUT RNA selectable marker bacterial replication-selection region has at least 95% sequence identity to a sequence selected from the group consisting of SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

In one embodiment, the present technology provides a method for eliminating antibiotic resistant marker gene transfer from a covalently closed circular non-viral transposon plasmid comprising the following steps: a) providing a covalently closed circular plasmid comprising: i) a 1 kb or larger bacterial replication-selection region comprising a Pol I-dependent origin of replication and an antibiotic resistance marker, and ii) an insert comprising an antibiotic resistance marker free eukaryotic region selected from the group consisting of non-viral transposon vector, non-viral transposase vector, Sleeping Beauty transposon vector, Sleeping Beauty transposase vector, PiggyBac transposon vector, PiggyBac transposase vector, Tol2 transposon vector, Tol2 transposase vector; and b) modifying the covalently closed circular recombinant molecule of a) such that the 1 kb or larger bacterial replication-selection region comprising a Pol I-dependent origin of replication and an antibiotic selectable marker is replaced with an less than 1 kb Pol III-dependent R6K origin-RNA-OUT RNA selectable marker bacterial replication-selection region, whereby the resultant Pol III-dependent origin of replication covalently closed circular plasmid has no antibiotic resistance markers that could be transposed into the genome when transfected into mammalian cells. In a further embodiment said Pol I-dependent origin of replication is selected from the group consisting of pUC origin, pMB1 origin, and ColE1 origin. In a further embodiment said Pol III-dependent R6K origin of replication is an R6K gamma replication origin with at least 95% sequence identity to a sequence selected from the group consisting of SEQ ID NO: 1, SEQ ID NO: 2, SEQ ID NO: 3, SEQ ID NO: 4 and SEQ ID NO: 18. In a further embodiment said RNA-OUT RNA selectable marker is an RNA-IN regulating RNA-OUT functional variant with at least 95% sequence identity to a sequence selected from the group consisting of SEQ ID NO: 5, and SEQ ID NO: 7. In a further embodiment said RNA-OUT RNA selectable marker is an RNA-OUT RNA selectable marker that encodes an RNA-IN regulating RNA-OUT RNA with at least 95% sequence identity to SEQ ID NO: 6. In a further embodiment said less than 1 kb Pol III-dependent R6K origin-RNA-OUT RNA selectable marker bacterial replication-selection region has at least 95% sequence identity to a sequence selected from the group consisting of SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

In one embodiment, the present technology provides an antibiotic marker free covalently closed circular recombinant DNA molecule comprising: a) an antibiotic marker free insert comprising a eukaryotic region selected from the group consisting of Lentiviral vector, Lentiviral envelope vector, Lentiviral packaging vector, Retroviral vector, Retroviral envelope vector, Retroviral packaging vector, AAV vector, AAV rep cap vector, Ad helper vector, Ad helper rep cap vector, non-viral transposon vector, and non-viral transposase vector; b) a Pol III-dependent origin of replication comprising an R6K gamma replication origin with at least 95% sequence identity to a sequence selected from the group consisting of SEQ ID NO: 1, SEQ ID NO: 2, SEQ ID NO: 3, SEQ ID NO: 4 and SEQ ID NO: 18; and c) an RNA-OUT RNA selectable marker comprising an RNA-IN regulating RNA-OUT functional variant with at least 95% sequence identity to a sequence selected from the group consisting of SEQ ID NO: 5, and SEQ ID NO: 7. In a further embodiment said R6K gamma replication origin and said RNA-OUT RNA selectable marker comprise a R6K origin-RNA-OUT RNA selectable marker bacterial replication-selection region with at least 95% sequence identity to a sequence selected from the group consisting of SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

In one embodiment, the present technology provides a method for reducing transfection associated toxicity from a covalently closed circular viral vector or non-viral transposon plasmid comprising: a) providing a covalently closed circular plasmid comprising: i) a 1 kb or larger bacterial replication-selection region comprising a Pol I-dependent origin of replication and an antibiotic selectable marker, and ii) an insert comprising a eukaryotic region selected from the group consisting of Lentiviral vector, Retroviral vector, AAV vector and non-viral transposon vector; modifying the covalently closed circular recombinant molecule of a) such that the 1 kb or larger bacterial replication-selection region comprising a Pol I-dependent origin of replication and an antibiotic selectable marker is replaced with an less than 1 kb Pol III-dependent R6K origin-RNA-OUT RNA selectable marker bacterial replication-selection region, whereby the resultant Pol III-dependent origin of replication covalently closed circular plasmid has reduced toxicity when transfected by transfection associated into mammalian cells. In a further embodiment said Pol I-dependent origin of replication is selected from the group consisting of pUC origin, pMB1 origin, and ColE1 origin. In a further embodiment said Pol III-dependent R6K origin of replication is an R6K gamma replication origin with at least 95% sequence identity to a sequence selected from the group consisting of SEQ ID NO: 1, SEQ ID NO: 2, SEQ ID NO: 3, SEQ ID NO: 4 and SEQ ID NO: 18. In a further embodiment said RNA-OUT RNA selectable marker is an RNA-IN regulating RNA-OUT functional variant with at least 95% sequence identity to a sequence selected from the group consisting of SEQ ID NO: 5, and SEQ ID NO: 7. In a further embodiment said RNA-OUT RNA selectable marker is an RNA-OUT RNA selectable marker that encodes an RNA-IN regulating RNA-OUT RNA with at least 95% sequence identity to SEQ ID NO: 6. In a further embodiment said less than 1 kb Pol III-dependent R6K origin-RNA-OUT RNA selectable marker bacterial replication-selection region has at least 95% sequence identity to a sequence selected from the group consisting of SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

The resultant Pol III-dependent replication origin plasmids have surprisingly improved manufacturing quality and yield than the parent pMB1, ColE1 or pBR322 derived replication origin expression plasmid vector.

Further objects and advantages of the invention will become apparent from a consideration of the drawings and ensuing description.

### BRIEF DESCRIPTION OF THE FIGURES

FIGS. 1A-1F depict the R6K origin (FIGS. 1A, 1E, and 1F), RNA-OUT selectable marker (FIG. 1B), and 14 and 3 CpG R6K-RNA-OUT bacterial backbones (FIGS. 1C and 1D);

FIGS. 2A-2B depict a Pol I-dependent pUC origin Sleeping Beauty transposon vector (FIG. 2A) and a Pol III-dependent R6K origin Sleeping Beauty transposon vector (FIG. 2B);

FIGS. 3A-3C depict Pol I-dependent pUC origin AAV vectors (FIGS. 3A and 3B) and a Pol III-dependent R6K origin AAV vectors (FIG. 3C); and

FIGS. 4A-4F depict Pol I-dependent pUC origin A60 polyA repeat encoding mRNA vectors (FIGS. 4A-4B), a Pol III-dependent R6K origin A60 polyA repeat encoding mRNA vector (FIG. 4C), Pol I-dependent pUC origin A99 polyA repeat encoding mRNA vectors (FIGS. 4D-4E), and a Pol III-dependent R6K origin A99 polyA repeat encoding mRNA vector (FIG. 4F).

Table 1: pVAX1 mammalian expression vector spacer region (SR) derivatives expression level
Table 2: NTC8685 mammalian expression vector spacer region (SR) derivatives expression level
Table 3: Minicircle applications with various viral and non-viral vector platforms
Table 4: pNTC multiple cloning site flanked R6K Origin-RNA-OUT selection marker vectors
Table 5: SV40 origin Lentiviral vectors: pUC versus R6K origin shake flask production yields/quality
Table 6: Sleeping Beauty Transposon vectors: pUC versus R6K origin shake flask production yields/quality
Table 7: AAV vectors: pUC versus R6K origin shake flask production yields/quality
Table 8: mRNA vectors: pUC versus R6K origin DH5a HyperGRO fermentation yields/quality
Table 9: AAV helper vectors: pUC versus R6K origin plasmid production yields/quality
SEQ ID NO:1: R6K gamma origin
SEQ ID NO:2: 1 CpG R6K gamma origin
SEQ ID NO:3: CpG free R6K gamma origin
SEQ ID NO:4: Extended R6K gamma origin
SEQ ID NO:5: RNA-OUT Selectable Marker
SEQ ID NO:6: RNA-OUT antisense repressor RNA
SEQ ID NO:7: 2 CpG RNA-OUT Selectable Marker
SEQ ID NO:8: R6K gamma origin-RNA-OUT bacterial region flanked by NheI and KpnI restriction sites
SEQ ID NO:9: 1 CpG R6K gamma origin-2 CpG RNA-OUT bacterial region flanked by NheI and KpnI restriction sites
SEQ ID NO:10: pNTC-NP1 polylinker trpA R6K-RNA-OUT polylinker cloning cassette: EcoRI/HindIII
SEQ ID NO:11: pNTC-NP2 polylinker trpA R6K-RNA-OUT polylinker cloning cassette: EcoRI/HindIII
SEQ ID NO:12: pNTC-NP3 polylinker trpA R6K-RNA-OUT polylinker cloning cassette: EcoRI/HindIII
SEQ ID NO:13: pNTC-NP4 polylinker trpA R6K-RNA-OUT polylinker cloning cassette: EcoRI/HindIII
SEQ ID NO:14: pNTC-NP5 polylinker trpA R6K-RNA-OUT polylinker cloning cassette: KasI/HindIII
SEQ ID NO:15: pNTC-NP6 polylinker trpA R6K-RNA-OUT polylinker cloning cassette: EcoRI/SacI
SEQ ID NO:16: pNTC-NP7 polylinker trpA R6K-RNA-OUT polylinker cloning cassette: BssHII-BssHII
SEQ ID NO:17: pNTC-3×CpG NP1 polylinker R6K-RNA-OUT polylinker cloning cassette: HindIII-EcoRI
SEQ ID NO:18: R6K gamma origin (7 iteron)
SEQ ID NO:19: R6K gamma origin 22 bp iteron repeat
SEQ ID NO:20: R6K gamma origin 22 bp iteron repeat
SEQ ID NO:21: R6K gamma origin 22 bp iteron repeat
SEQ ID NO:22: R6K gamma origin 22 bp iteron repeat
SEQ ID NO:23: R6K gamma origin 22 bp iteron repeat

### DEFINITION OF TERMS

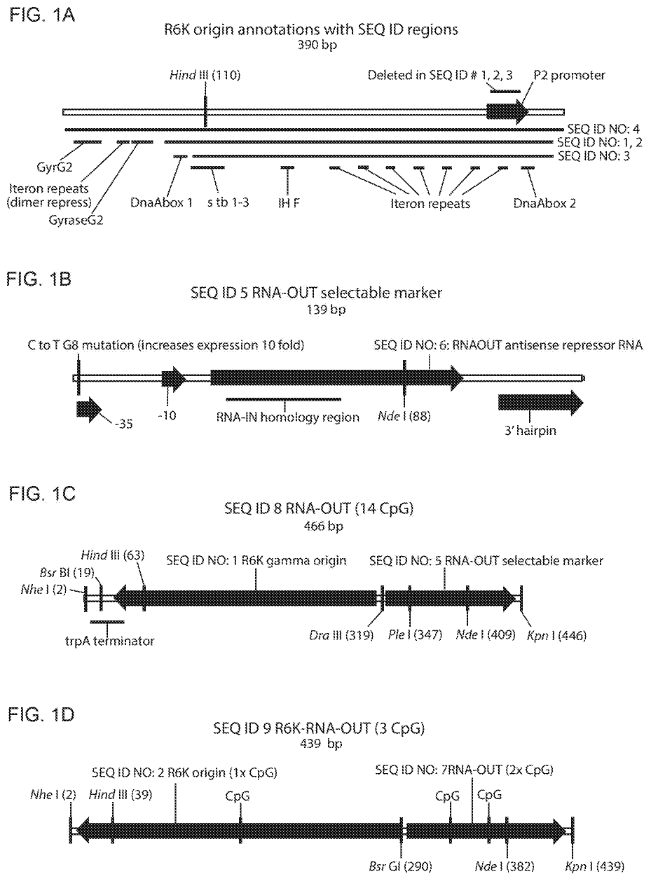

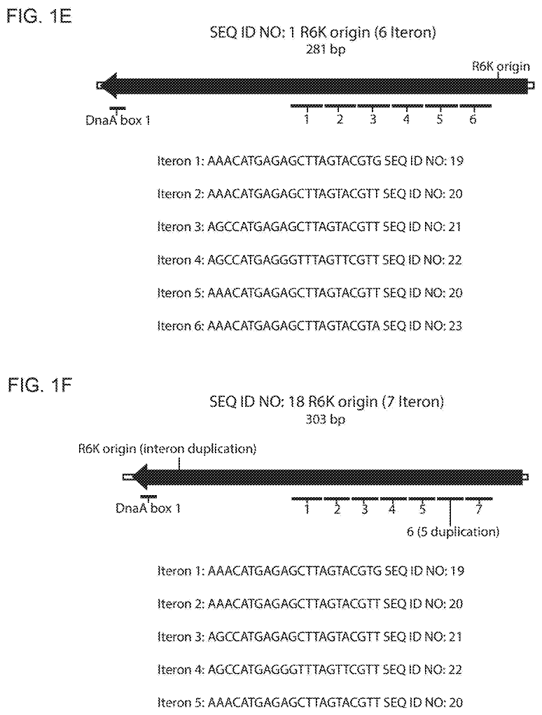

AAV vector: Adeno-associated virus vector, an episomal viral vector. Includes self-complementary (sc) Adeno-associated virus vectors (scAAV) and single-stranded (ss) Adeno-associated virus vectors (ssAAV)
AF: Antibiotic-free
amp: Ampicillin
ampR: Ampicillin Resistance gene
Antibiotic selectable marker: A gene that confirs resistance to an antibiotic, e.g. ampicillin resistance gene, kanamycin resistance gene, chloramphenicol resistance gene, tetracycline resistance gene
Approximately: As used herein, the term “approximately” or “about,” as applied to one or more values of interest, refers to a value that is the same or similar to a stated reference value
Bacterial region: Region of a plasmid vector required for propagation and selection in the bacterial host
bp: basepairs
ccc: Covalently Closed Circular
cI: Lambda repressor
cITs857: Lambda repressor further incorporating a C to T (Ala to Thr) mutation that confers temperature sensitivity. cITs857 is a functional repressor at 28-30° C. but is mostly inactive at 37-42° C. Also called cI857
Cat^{R}: Chloramphenicol resistance gene
cmv: Cytomegalovirus
dcm methylation: E. coli methyltransferase that metiylated the sequences CC(A/T)GG at the C5 position of the second cytosine
DNA replicon: A genetic element that can replicate under its own control; examples include plasmids, cosmids, bacterial artificial chromosomes (BACs), bacteriophages, viral vectors and hybrids thereof
E. coli: Escherichia coli, a gram negative bacteria
EGFP: Enhanced green fluorescent protein
EP: Electroporation
Eukaryotic expression vector: A vector for expression of mRNA, protein antigens, protein therapeutics, shRNA, RNA or microRNA genes in a target eukaryotic organism using RNA Polymerase I, II or III promoters
Eukaryotic region: The region of a plasmid that encodes eukaryotic sequences and/or sequences required for plasmid function in the target organism. This includes the region of a plasmid vector required for expression of one or more transgenes in the target organism including RNA Pol II enhancers, promoters, transgenes and polyA sequences. This also includes the region of a plasmid vector required for expression of one or more transgenes in the target organism using RNA Pol I or RNA Pol III promoters, RNA Pol I or RNA Pol III expressed transgenes or RNAs. The eukaryotic region may optionally include other functional sequences, such as eukaryotic transcriptional terminators, supercoiling-induced DNA duplex destabilized (SIDD) structures, S/MARs, boundary elements, etc. In a Lentiviral or Retroviral vector, the eukaryotic region contains flanking direct repeat LTRs, in a AAV vector the eukaryotic region contains flanking inverted terminal repeats, while in a Transposon vector the eukaryotic region contains flanking transposon inverted terminal repeats or IR/DR termini (e.g. Sleeping Beauty). In genome integration vectors, the eukaryotic region may encode homology arms to direct targeted integration
Exon: A nucleotide sequence encoded by a gene that is transcribed and present within a mature mRNA product after RNA splicing to remove introns has been completed
Expression vector: A vector for expression of mRNA, protein antigens, protein therapeutics, shRNA, RNA or microRNA genes in a target organism.
g: Gram, kg for kilogram
gene of interest: gene to be expressed in the target organism. Includes mRNA genes that encode protein or peptide antigens, protein or peptide therapeutics, and mRNA, shRNA, RNA or microRNA that encode RNA therapeutics, and mRNA, shRNA, RNA or microRNA that encode RNA vaccines, etc.
Hr(s): Hour(s)
ID: Intradermal
IM: Intramuscular
immune response: Antigen reactive cellular (e.g. antigen reactive T cells) or antibody (e.g. antigen reactive IgG) responses
Intron: A nucleotide sequence encoded by a gene that is transcribed and subsequently removed from a mature mRNA product by RNA splicing
IR/DR: Inverted Repeats which are each Directly Repeated twice. For example, Sleeping Beauty transposon IR/DR repeats
Iteron: Directly repeated DNA sequences in a origin of replication that are required for replication initiation. R6K origin iteron repeats are 22 bp
ITR: Inverted Terminal Repeat
kan: Kanamycin
kanR: Kanamycin Resistance gene
Kd: Kilodalton
kozak sequence: Optimized consensus DNA sequence gccRccATG (R=G or A) immediately upstream of an ATG start codon that ensures efficient tranlation initiation. A SalI site (GTCGAC) immediately upstream of the ATG start codon (GTCGACATG) is an effective kozak sequence
Lentiviral vector: Integrative viral vector that can infect dividing and non-dividing cells. Also call Lentiviral transfer plasmid. Plasmid encodes Lentiviral LTR flanked expression unit. Transfer plasmid is transfected into production cells along with Lentiviral envelope and packaging plasmids required to make viral particles
Lentiviral envelope vector: Plasmid encoding envelope glycoprotein
Lentiviral packaging vector: One or two plasmids that express gag, pol and Rev functions required to package the Lentiviral transfer vector
minicircle: Covalently closed circular plasmid derivatives in which the bacterial region has been removed from the parent plasmid by in vivo or in vitro site-specific recombination or in vitro restriction digestion/ligation. Minicircle vectors are replication incompetent in bacterial cells
mRNA: Messenger RNA
mSEAP: Murine secreted alkaline phosphatase
NA: Not Applicable
Nanoplasmid™ vector: Vector combining an RNA selectable marker with a R6K, ColE2 or ColE2 related replication origin. For example, NTC9385C, NTC9685C, NTC9385R, NTC9685R vectors and modifications described in Williams, Supra, 2014 and included herein by reference
NTC8385: NTC8385, NTC8485 and NTC8685 plasmids are antibiotic-free pUC origin vectors that contain a short RNA (RNA-OUT) selectable marker instead of an antibiotic resistance marker such as kanR. The creation and application of these RNA-OUT based antibiotic-free vectors are described in Williams, J A 2008 World Patent Application WO2008153733 and included herein by reference
NTC8485: NTC8485 is an antibiotic-free pUC origin vector that contains a short RNA (RNA-OUT) selectable marker instead of an antibiotic resistance marker such as kanR. The creation and application of NTC8485 is described in Williams, J A 2010 US Patent Application 20100184158 and included herein by reference
NTC8685: NTC8685 is an antibiotic-free pUC origin vector that contains a short RNA (RNA-OUT) selectable marker instead of an antibiotic resistance marker such as kanR. The creation and application of NTC8685 is described in Williams, Supra, 2010 and included herein by reference
NTC9385R: The NTC9385R Nanoplasmid™ vector described in Williams, Supra, 2014 included herein by reference has a spacer region encoded NheI-trpA terminator-R6K origin RNA-OUT-KpnI bacterial region (SEQ ID NO:8) linked through the flanking NheI and KpnI sites to the eukaryotic region.
OD₆₀₀: optical density at 600 nm
PAS: Primosomal assembly site. Priming of DNA synthesis on a single stranded DNA ssi site. ØX174 type PAS: DNA hairpin sequence that binds priA, which, in turn, recruits the remaining proteins to form the preprimosome [priB, dnaT, recruits dnaB (delivered by dnaC)], which then also recruits primase (dnaG), which then, finally, makes a short RNA substrate for DNA polymerase I. ABC type PAS: DNA hairpin binds dnaA, recruits dnaB (delivered by dnaC) which then also recruits primase (dnaG), which then, finally, makes a short RNA substrate for DNA polymerase I. For example, the R6K plasmid CpG free ssiA primosomal assembly site or alternative OX174 type or ABC type primosomal assembly sites
PAS-BH: Primosomal assembly site on the heavy (leading) strand
PAS-BH region: pBR322 origin region between ROP and PAS-BL (approximately pBR322 2067-2351)
PAS-BL: Primosomal assembly site on the light (lagging) strand
PBS: Phosphate buffered Saline
PCR: Polymerase Chain Reaction
pDNA: Plasmid DNA
PiggyBac Transposon: PB transposon. A transposon system that integrates an ITR flanked PB transposon into the genome by a simple cut and paste mechanism mediated by PB transposase. The transposon vector typically contains a promoter-transgene-polyA expression cassette between the PB ITRs which is excised and integrated into the genome
pINT pR pL vector: The pINT pR pL att_{HK022} integration expression vector is described in Luke et al., 2011 Mol Biotechnol 47:43 and included herein by reference. The target gene to be expressed is cloned downstream of the pL promoter. The vector encodes the temperature inducible cI857 repressor, allowing heat inducible target gene expression
P_{L} promoter: Lambda promoter left. P_{L} is a strong promoter that is repressed by the cI repressor binding to OL1, OL2 and OL3 repressor binding sites. The temperature sensitive cI857 repressor allows control of gene expression by heat induction since at 30° C. the cI857 repressor is functional and it represses gene expression, but at 37-42° C. the repressor is inactivated so expression of the gene ensues
P_{L} (OL1 G to T) promoter: Lambda promoter left. P_{L} is a strong promoter that is repressed by the cI repressor binding to OL1, OL2 and OL3 repressor binding sites. The temperature sensitive c1857 repressor allows control of gene expression by heat induction since at 30° C. the c1857 repressor is functional and it represses gene expression, but at 37-42° C. the repressor is inactivated so expression of the gene ensues. The cI repressor binding to OL1 is reduced by the OL1 G to T mutation resulting in increased promoter activity at 30° C. and 37-42° C. as described in Williams, Supra, 2014.
Plasmid: An extra chromosomal DNA molecule separate from the chromosomal DNA which is capable of replicating independently from the chromosomal DNA
Plasmid copy number: the number of copies of a plasmid per cell. Increases in plasmid copy number increase plasmid production yield
Pol: Polymerase
Pol I: Escherichia coli DNA Polymerase I
Pol I dependent origin of replication: A replication origin that requires Pol I, for example the pMB1, ColE1 or pBR322 or derivatives such as the high copy pUC origin. For these origins the RNAII primer forms an RNA: DNA R-loop that is cleaved by RNase H to create a primer for DNA pol I directed DNA synthesis. DNA synthesis then converts to DNA pol III. Numerous additional Pol I dependent replication origins are known in the art, many of which are summarized in del Solar et al., 1998 Microbiol. Mol. Biol. Rev 62:434-464 which is included herein by reference
Pol III: Escherichia co/i DNA Polymerase III
Pol III dependent origin of replication: A replication origin that doesn't require Pol I, for example the rep protein dependent R6K gamma replication origin. Numerous additional Pol III dependent replication origins are known in the art, many of which are summarized in del Solar et al., Supra, 1998 which is included herein by reference
polyA: Polyadenylation signal or site. Polyadenylation is the addition of a poly(A) tail to an RNA molecule. The polyadenylation signal contains the sequence motif recognized by the RNA cleavage complex. Most human polyadenylation signals contain an AAUAAA motif and conserved sequences 5′ and 3′ to it. Commonly utilized polyA signals are derived from the rabbit β globin, bovine growth hormone, SV40 early, or SV40 late polyA signals
pUC origin: pBR322-derived replication origin, with G to A transition that increases copy number at elevated temperature and deletion of the ROP negative regulator
pUC free: Plasmid that does not contain the pUC origin. Non-replicative fragments of the pUC origin may be included, for example the RNAI selectable marker
pUC plasmid: Plasmid containing the pUC origin
R6K plasmid: NTC9385R, NTC9685R, NTC9385R2-O1, NTC9385R2-O2, NTC9385R2a-O1, NTC9385R2a-O2, NTC9385R2b-O1, NTC9385R2b-O2, NTC9385Ra-O1, NTC9385Ra-O2, NTC9385RaF, and NTC9385RbF vectors as well as modifications and alternative vectors containing a R6K replication origin that were described in Williams, Supra, 2014 and included herein by reference. Alternative R6K vectors known in the art including, but not limited to, pCOR vectors (Gencell), pCpGfree vectors (Invivogen), and CpG free University of Oxford vectors including pGM169
R6K replication origin: a region which is specifically recognized by the R6K Rep protein to initiate DNA replication. Includes but not limited to R6K gamma replication origin sequence disclosed as SEQ ID NO:1, SEQ ID NO:2 SEQ ID NO:4, and SEQ ID NO:18. Also includes CpG free versions (e.g. SEQ ID NO:3) as described in Drocourt et al., U.S. Pat. No. 7,244,609 and incorporated herein by reference
R6K replication origin-RNA-OUT bacterial region: Contains a R6K replication origin for propagation and the RNA-OUT selectable marker (e.g. SEQ ID NO:8; SEQ ID NO:9; SEQ ID NO:10; SEQ ID NO:11; SEQ ID NO:12; SEQ ID NO:13; SEQ ID NO: 14; SEQ ID NO: 15; SEQ ID NO: 16; SEQ ID NO: 17)
Rep: Replication
Replication intermediates: Linear DNA fragments resulting from premature termination of plasmid replication
Rep protein dependent plasmid: A plasmid in which replication is dependent on a replication (Rep) protein provided in Trans. For example, R6K replication origin, ColE2-P9 replication origin and ColE2 related replication origin plasmids in which the Rep protein is expressed from the host strain genome. Numerous additional Rep protein dependent plasmids are known in the art, many of which are summarized in del Solar et al., Supra, 1998 which is included herein by reference
Retroviral vector: Integrative viral vector that can infect dividing cells. Also call transfer plasmid. Plasmid encodes Retroviral LTR flanked expression unit. Transfer plasmid is transfected into production cells along with envelope and packaging plasmids required to make viral particles
Retroviral envelope vector: Plasmid encoding envelope glycoprotein
Retroviral packaging vector: Plasmid that encodes Retroviral gag, pol genes required to package the Retroviral transfer vector
RNA-IN: Insertion sequence 10 (IS10) encoded RNA-IN, an RNA complementary and antisense to a portion of RNA RNA-OUT. When RNA-IN is cloned in the untranslated leader of a mRNA, annealing of RNA-IN to RNA-OUT reduces translation of the gene encoded downstream of RNA-IN
RNA-IN regulated selectable marker: A genomically expressed RNA-IN regulated selectable marker. In the presence of plasmid borne RNA-OUT antisense repressor RNA (SEQ ID NO:6), expression of a protein encoded downstream of RNA-IN is repressed. An RNA-IN regulated selectable marker is configured such that RNA-IN regulates either 1) a protein that is lethal or toxic to said cell per se or by generating a toxic substance (e.g. SacB), or 2) a repressor protein that is lethal or toxic to said bacterial cell by repressing the transcription of a gene that is essential for growth of said cell (e.g. murA essential gene regulated by RNA-IN tetR repressor gene). For example, genomically expressed RNA-IN-SacB cell lines for RNA-OUT plasmid selection/propagation are described in Williams, Supra, 2008 and included herein by reference. Alternative selection markers described in the art may be substituted for SacB
RNA-OUT: Insertion sequence 10 (IS10) encoded RNA-OUT, an antisense RNA that hybridizes to, and reduces translation of, the transposon gene expressed downstream of RNA-IN. The sequence of the RNA-OUT RNA (SEQ ID NO:6) and complementary RNA-IN SacB genomically expressed RNA-IN-SacB cell lines can be modified to incorporate alternative functional RNA-IN/RNA-OUT binding pairs such as those described in Mutalik et al., 2012 Nat Chem Biol 8:447, including, but not limited to, the RNA-OUT A08/RNA-IN S49 pair, the RNA-OUT A08/RNA-IN S08 pair, and CpG free modifications of RNA-OUT A08 that modify the CG in the RNA-OUT 5′ TTCGC sequence to a non-CpG sequence. An example of a CpG free RNA-OUT selection marker, in which the two CpG motifs in the RNA-OUT RNA (one of which is present in the RNA-IN complementary region) are removed, was described in Williams 2015. Replicative minicircle vectors with improved expression. US Patent Application US 2015/0275221 and included herein by reference. A multitude of alternative substitutions to remove the two CpG motifs (mutating each CpG to either CpA, CpC, CpT, ApG, GpG, or TpG) may be utilized to make a CpG free RNA-OUT
RNA-OUT Selectable marker: An RNA-OUT selectable marker DNA fragment including E. coli transcription promoter and terminator sequences flanking an RNA-OUT RNA. An RNA-OUT selectable marker, utilizing the RNA-OUT promoter and terminator sequences, that is flanked by DraIII and KpnI restriction enzyme sites, and designer genomically expressed RNA-IN-SacB cell lines for RNA-OUT plasmid propagation, are described in Williams, Supra, 2008 and included herein by reference. The RNA-OUT promoter and terminator sequences in SEQ ID NO: 5 that flank the RNA-OUT RNA (SEQ ID NO:6; FIG. 1B) may be replaced with heterologous promoter and terminator sequences. For example, the RNA-OUT promoter may be substituted with a CpG free promoter known in the art, for example the I-EC2K promoter or the P5/6 5/6 or P5/6 6/6 promoters described in Williams, Supra, 2008 and included herein by reference. A 2 CpG RNA-OUT selectable marker in which the two CpG motifs in the RNA-OUT promoter are removed is given as SEQ ID NO: 7. An example of a CpG free RNA-OUT transcription unit, in which the two CpG motifs in the RNA-OUT RNA (one of which is present in the RNA-IN complementary region) and the two CpG motifs in the RNA-OUT promoter are removed was described in Williams, Supra, 2015 and included herein by reference. Vectors incorporating CpG free RNA-OUT selectable marker may be selected for sucrose resistance using the RNA-IN-SacB cell lines for RNA-OUT plasmid propagation described in Williams, Supra, 2008. Alternatively, the RNA-IN sequence in these cell lines can be modified to incorporate the 1 bp change needed to perfectly match the CpG free RNA-OUT region complementary to RNA-IN.
RNA polymerase II promoter: Promoter that recruits RNA Polymerase II to synthesize mRNAs, most small nuclear RNAs and microRNAs. For example, constitutive promoters such as the human or murine CMV promoter, elongation factor 1 (EF1) promoter, the chicken β-actin promoter, the β-actin promoter from other species, the elongation factor-1 α (EF1 α) promoter, the phosphoglycerokinase (PGK) promoter, the Rous sarcoma virus (RSV) promoter, the human serum albumin (SA) promoter, the spleen focus-forming virus (SFFV) promoter, the α-1 antitrypsin (AAT) promoter, the thyroxine binding globulin (TBG) promoter, the cytochrome P450 2E1 (CYP2E1) promoter, etc. The vectors may also utilize combination promoters such as the chicken β-actin/CMV enhancer (CAG) promoter, the human or murine CMV-derived enhancer elements combined with the elongation factor 1α (EF1α) promoters, CpG free versions of the human or murine CMV-derived enhancer elements combined with the elongation factor 1α (EF1α) promoters, the albumin promoter combined with an α-fetoprotein MERII enhancer, etc., or the diversity of tissue specific or inducible promoters know in the art such as the muscle specific promoters muscle creatine kinase (MCK), and C5-12 or the liver-specific promoter apolipoprotein A-I (ApoAI), etc.
RNA polymerase III promoter: Promoter that recruits RNA Polymerase III to synthesize tRNAs, 5S ribosomal RNA, and other small RNAs. For example, Class I promoters such as the 5s rRNA promoter, Class II promoter such as tRNA promoters, Class III promoters such as the U6 small nuclear RNA promoter or the H1 nuclear RNase P promoter, etc.
RNA selectable marker: An RNA selectable marker is a plasmid borne expressed non-translated RNA that regulates a chromosomally expressed target gene to afford selection. This may be a plasmid borne nonsense suppressing tRNA that regulates a nonsense suppressible selectable chromosomal target as described by Crouzet J and Soubrier F 2005 U.S. Pat. No. 6,977,174 included herein by reference. This may also be a plasmid borne antisense repressor RNA, a non limiting list included herein by reference includes RNA-OUT that represses RNA-IN regulated targets (Williams, Supra, 2008), pMB1 plasmid origin encoded RNAI that represses RNAII regulated targets (Grabherr R, Pfaffenzeller I. 2006 US patent application US20060063232; Cranenburgh R M. 2009; U.S. Pat. No. 7,611,883), IncB plasmid pMU720 origin encoded RNAI that represses RNA II regulated targets (Wilson I W, Siemering K R, Praszkier J, Pittard A J. 1997. J Bacteriol 179:742-53), ParB locus Sok of plasmid R1 that represses Hok regulated targets, Flm locus FlmB of F plasmid that represses fimA regulated targets (Morsey M A, 1999 US patent U.S. Pat. No. 5,922,583). An RNA selectable marker may be another natural antisense repressor RNAs known in the art such as those described in Wagner E G H, Altuvia S, Romby P. 2002. Adv Genet 46:361-98 and Franch T, and Gerdes K. 2000. Current Opin Microbiol 3:159-64. An RNA selectable marker may also be an engineered repressor RNAs such as synthetic small RNAs expressed SgrS, MicC or MicF scaffolds as described in Na D, Yoo S M, Chung H, Park H, Park J H, Lee S Y. 2013. Nat Biotechnol 31:170-4. An RNA selectable marker may also be an engineered repressor RNA as part of a selectable marker that represses a target RNA fused to a target gene to be regulated such as SacB as described in Williams, Supra, 2015
ROP: Repressor of primer
RSM: RNA selectable marker
SacB: Structural gene encoding Bacillus subtilis levansucrase. Expression of SacB in gram negative bacteria is toxic in the presence of sucrose
SD: Standard deviation
SEAP: Secreted alkaline phosphatase
Selectable marker: A selectable marker, for example a kanamycin resistance gene or an RNA selectable marker
Selection marker: A selectable marker, for example a kanamycin resistance gene or an RNA selectable marker
SIDD: supercoiling-induced DNA duplex destabilized (SIDD) structures. These sites, when incorporated into a vector, may alter the susceptibility of other sequences within the vector to be destabilized. This can alter function. For example, addition of a SIDD site to an expression vector may reduce the helical destabilization of a promoter. This may increase or decrease promoter activity, depending on the promoter since some promoters have increased expression with promoter helical destabilization, while others will have reduced expression with promoter helical destabilization
shRNA: Short hairpin RNA
S/MAR: Scaffold/matrix attached region. Eukaryotic sequences that mediate DNA attachment to the nuclear matrix
Sleeping Beauty Transposon: SB transposon. A transposon system that integrates an IR/DR flanked SB transposon into the genome by a simple cut and paste mechanism mediated by SB transposase. The transposon vector typically contains a promoter-transgene-polyA expression cassette between the IR/DRs which is excised and integrated into the genome
Spacer region: As used herein, spacer region is the region linking the 5′ and 3′ ends of the eukaryotic region sequences. The eukaryotic region 5′ and 3′ ends are typically separated by the bacterial replication origin and bacterial selectable marker in plasmid vectors (bacterial region) so many spacer regions consist of the bacterial region. In Pol III dependent origin of replication vectors of the invention, this spacer region preferably is less than 1000 bp
SR: Spacer region.
ssi: Single stranded initiation sequences
Structured DNA sequence: As used herein, a DNA sequence that is capable of forming replication inhibiting secondary structures (Mirkin and Mirkin, 2007. Microbiology and Molecular Biology Reviews 71:13-35). This includes but is not limited to inverted repeats, palindromes, direct repeats, IR/DRs, homopolymeric repeats or repeat containing eukaryotic promoter enhancers, or repeat containing eukaryotic origin of replications.
SV40 origin: Simian Virus 40 genomic DNA that contains the origin of replication
SV40 enhancer: Simian Virus 40 genomic DNA that contains the 72 bp and optionally the 21 bp enhancer repeats
target antigen: Immunogenic protein or peptide epitope, or combination of proteins and epitopes, against which an immune response can be mounted. Target antigens may by derived from a pathogen for infectious disease or allergy applications or derived from a host organism for applications such as cancer, allergy, or autoimmune diseases. Target antigens are well defined in the art. Some examples are described in Williams, Supra, 2008 and are included herein by reference
TE buffer: A solution containing approximately 10 mM Tris pH 8 and 1 mM EDTA
TetR: Tetracycline resistance gene
Tol2 Transposon: A transposon system that integrates an ITR flanked Tol2 transposon into the genome by a simple cut and paste mechanism mediated by Tol2 transposase. The transposon vector typically contains a promoter-transgene-polyA expression cassette between the Tol2 ITRs which is excised and integrated into the genome
Transcription terminator: Bacterial: A DNA sequence that marks the end of a gene or operon for transcription. This may be an intrinsic transcription terminator or a Rho-dependent transcriptional terminator. For an intrinsic terminator, such as the trpA terminator, a hairpin structure forms within the transcript that disrupts the mRNA-DNA-RNA polymerase ternary complex. Alternatively, Rho-dependent transcriptional terminators require Rho factor, an RNA helicase protein complex, to disrupt the nascent mRNA-DNA-RNA polymerase ternary complex. Eukaryotic: PolyA signals are not ‘terminators’, instead internal cleavage at PolyA sites leaves an uncapped 5′end on the 3′UTR RNA for nuclease digestion. Nuclease catches up to RNA Pol II and causes termination. Termination can be promoted within a short region of the poly A site by introduction of RNA Pol II pause sites (eukaryotic transcription terminator). Pausing of RNA Pol II allows the nuclease introduced into the 3′ UTR mRNA after PolyA cleavage to catch up to RNA Pol II at the pause site. A nonlimiting list of eukaryotic transcription terminators know in the art include the C2×4 and the gastrin terminator. Eukaryotic transcription terminators may elevate mRNA levels by enhancing proper 3′-end processing of mRNA
transfection: Method to deliver nucleic acids into cells [e.g. poly(lactide-co-glycolide) (PLGA), ISCOMs, liposomes, niosomes, virosomes, block copolymers, Pluronic block copolymers, chitosan, and other biodegradable polymers, microparticles, microspheres, calcium phosphate nanoparticles, nanoparticles, nanocapsules, nanospheres, poloxamine nanospheres, electroporation, nucleofection, piezoelectric permeabilization, sonoporation, iontophoresis, ultrasound, SQZ high speed cell deformation mediated membrane disruption, corona plasma, plasma facilitated delivery, tissue tolerable plasma, laser microporation, shock wave energy, magnetic fields, contactless magneto-permeabilization, gene gun, microneedles, microdermabrasion, hydrodynamic delivery, high pressure tail vein injection, etc] as known in the art and included herein by reference
Transgene: Gene of interest that is cloned into a vector for expression in a target organism
Transposase vector: A vector which encodes a transposase
Transposon vector: A vector which encodes a transposon which is a substrate for transposase mediated gene integration
ts: Temperature sensitive
μg: Microgram
μl: Microliter
UTR: Untranslated region of a mRNA (5′ or 3′ to the coding region)
Vector: A gene delivery vehicle, including viral (e.g. Alphavirus, Poxvirus, Lentivirus, Retrovirus, Adenovirus, Adenovirus related virus, etc.) and non-viral (e.g. plasmid, MIDGE, transcriptionally active PCR fragment, minicircles, bacteriophage, etc.) vectors. These are well known in the art and are included herein by reference
Vector backbone: Eukaryotic region and bacterial region of a vector, without the transgene or target antigen coding region

### DETAILED DESCRIPTION OF THE PREFERRED EMBODIMENTS

The current technology relates generally to short <1 kb bacterial region plasmid DNA vector methods and compositions that improve plasmid manufacture yield and quality, reduce transfection associated toxicity, and increase transgene expression. The current technology can be practiced to improve expression and manufacturing of vectors such as non-viral vectors (transposon vector, transposase vector, Sleeping Beauty transposon vector, Sleeping Beauty transposase vector, PiggyBac transposon vector, PiggyBac transposase vector, expression vector, etc.) and viral vectors (e.g. AAV vector, AAV rep cap vector, AAV helper vector, Ad helper vector, Lentivirus vector, Lentiviral envelope vector, Lentiviral packaging vector, Retroviral vector, Retroviral envelope vector, Retroviral packaging vector, etc.).

Improved plasmid expression is defined herein as improved transgene expression level and/or expression duration in vitro or in vivo compared to a transgene encoding plasmid containing a bacterial region encoding the pUC replication origin. It is to be understood that all references cited herein are incorporated by reference in their entirety.

The methods of plasmid modification of the present current technology have been surprisingly found to provide a solution to provide short spacer region vectors containing structured DNA sequences with efficient high yield manufacture.

As used herein, the term “sequence identity” refers to the degree of identity between any given query sequence, e.g. SEQ ID NO: 2, and a subject sequence. A subject sequence may, for example, have at least 90 percent, at least 95 percent, or at least 99 percent sequence identity to a given query sequence. To determine percent sequence identity, a query sequence (e.g. a nucleic acid sequence) is aligned to one or more subject sequences using any suitable sequence alignment program that is well known in the art, for instance, the computer program ClustalW (version 1.83, default parameters), which allows alignments of nucleic acid sequences to be carried out across their entire length (global alignment). Chema et al., 2003 Nucleic Acids Res., 31:3497-500. In a preferred method, the sequence alignment program (e.g. ClustalW) calculates the best match between a query and one or more subject sequences, and aligns them so that identities, similarities, and differences can be determined. Gaps of one or more nucleotides can be inserted into a query sequence, a subject sequence, or both, to maximize sequence alignments. For fast pair-wise alignments of nucleic acid sequences, suitable default parameters can be selected that are appropriate for the particular alignment program. The output is a sequence alignment that reflects the relationship between sequences. To further determine percent identity of a subject nucleic acid sequence to a query sequence, the sequences are aligned using the alignment program, the number of identical matches in the alignment is divided by the length of the query sequence, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

Turning now to the drawings, FIGS. 1A-1F show annotated maps of FIG. 1A) R6K origin with the locations of the 22 bp iteron repeats, DnaA boxes 1 and 2, and the regions included in the SEQ ID NO: 1, 2, 3, and 4 R6K origins; FIG. 1B) SEQ ID NO: 5 RNA-OUT selectable marker with the locations of the RNA-OUT promoter-35 and -10 elements, SEQ ID NO: 6 RNA OUT antisense RNA with RNA-IN complementary homology region and RNA-OUT terminator 3′ hairpin; FIG. 1C) 14 CpG R6K-RNA-OUT bacterial backbone composed of SEQ ID NO: 1 R6K replication origin and SEQ ID NO: 5 RNA-OUT selectable marker including the trpA bacterial terminator upstream of the R6K origin and flanked by NheI and KpnI cloning sites; FIG. 1D) 3 CpG R6K-RNA-OUT bacterial backbone composed of SEQ ID NO: 2 1× CpG R6K replication origin and SEQ ID NO: 7 2×CpG RNA-OUT selectable marker flanked by NheI and KpnI cloning sites; FIG. 1E) R6K origin from SEQ ID NO: 1, with locations of the 6 iterons highlighted. The individual 22 bp iteron repeat sequences are shown below the origin map; and FIG. 1F) R6K origin from SEQ ID NO: 18, with locations of the 7 iterons highlighted. The individual 22 bp iteron repeat sequences are shown below the origin map. In this example 7 iteron vector iteron 5 has been tandemly duplication; however, a 7 iteron vector of the invention can be obtained by tandem duplication of any of iterons 1, 2, 3, 4, 5 or 6.

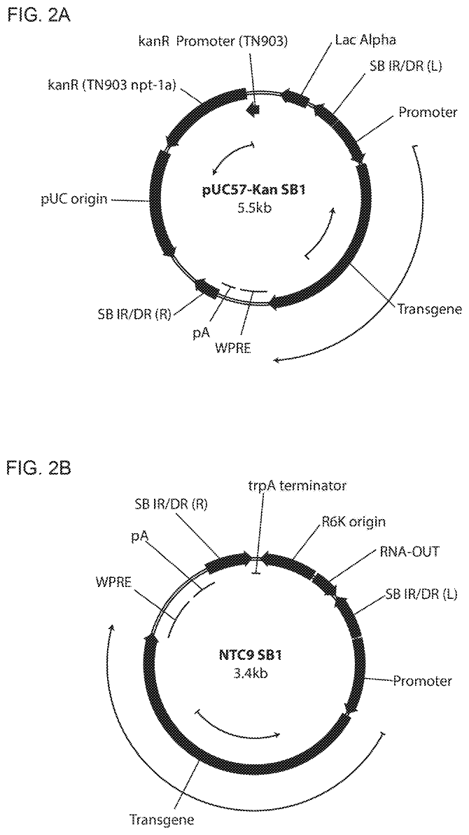

FIGS. 2A-2B show annotated maps of: FIG. 2A) Pol I-dependent pUC origin-Kanamycin selection Sleeping Beauty transposon vector pUC57-Kan SB1 (see Table 6); and FIG. 2B) Pol III-dependent R6K origin-RNA-OUT antibiotic free selection Sleeping Beauty transposon vector NTC9 SB1 (see Table 6). The locations of the left and right Sleeping Beauty IR/DR relative to the bacterial backbone replication origins and selection markers are shown.

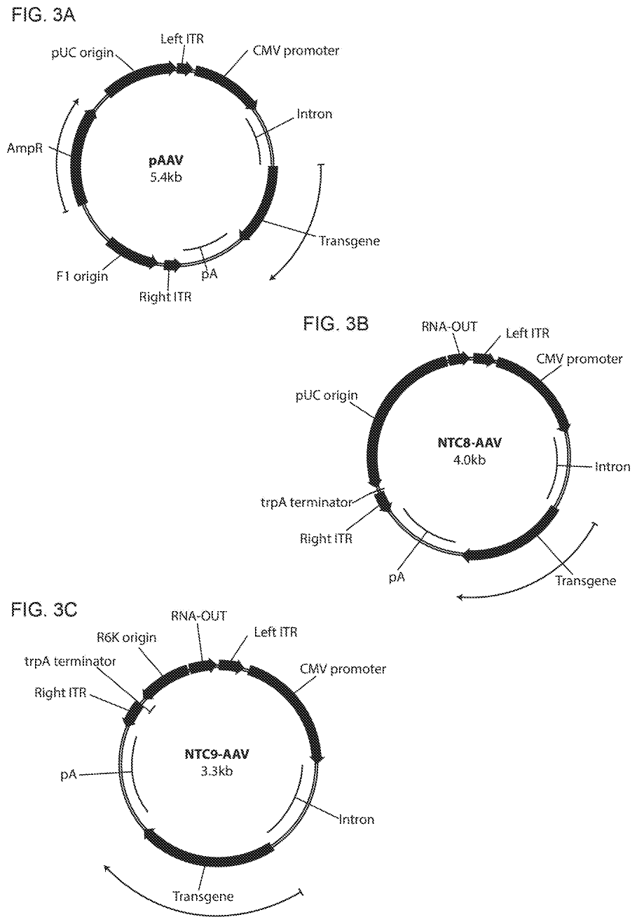

FIGS. 3A-3C show annotated maps of: FIG. 3A) Pol I-dependent pUC origin-Ampicillin selection AAV vector pAAV (see Table 7); FIG. 3B) Pol I-dependent pUC origin-RNA-OUT antibiotic free selection AAV vector NTC8-AAV (see Table 7); and FIG. 3C) Pol III-dependent R6K origin-RNA-OUT antibiotic free selection AAV vector NTC9-AAV (see Table 7). The locations of the left and right AAV ITRs relative to the bacterial backbone replication origins and selection markers are shown.

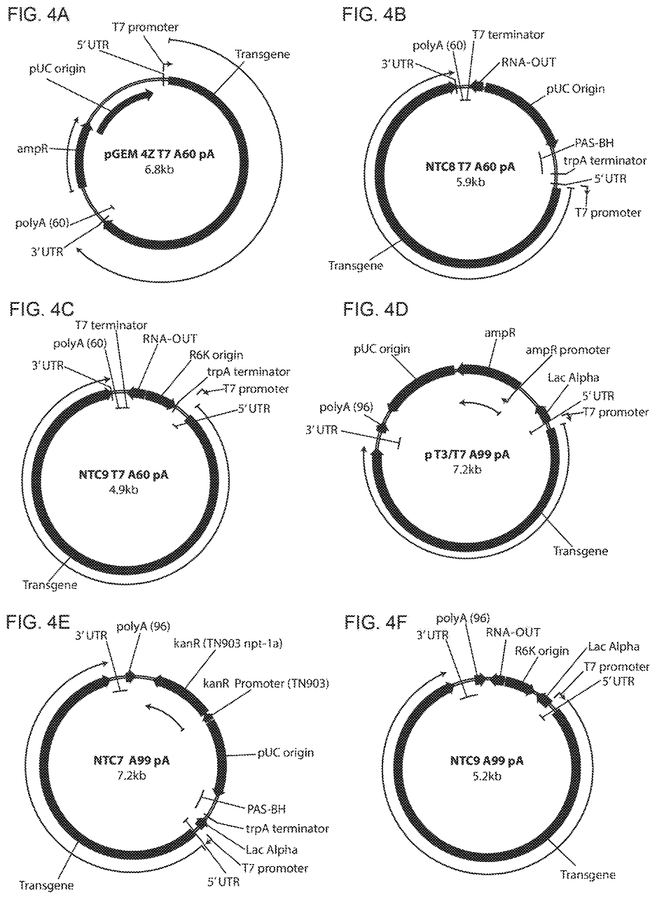

FIGS. 4A-4F show annotated maps of FIG. 4A) Pol I-dependent pUC origin-Ampicillin selection A60 polyA repeat encoding mRNA vector pGEM4Z T7 A60 pA (see Table 8); FIG. 4B) Pol I-dependent pUC origin-RNA-OUT antibiotic free selection A60 polyA repeat encoding mRNA vector NTC8-T7 A60 pA (see Table 8); FIG. 4C) Pol III-dependent R6K origin-RNA-OUT antibiotic free selection A60 polyA repeat encoding mRNA vector NTC9-T7 A60 pA (see Table 8); FIG. 4D) Pol I-dependent pUC origin-Ampicillin selection A99 polyA repeat encoding mRNA vector pT3/T7 A99 pA (see Table 8); FIG. 4E) Pol I-dependent pUC origin-kanR selection A99 polyA repeat encoding mRNA vector NTC7-T7 A99 pA (see Table 8); and FIG. 4F) Pol III-dependent R6K origin-RNA-OUT antibiotic free selection A99 polyA repeat encoding mRNA vector NTC9-T7 A99 pA (see Table 8). The location of the A60 or A99 polyA repeat relative to the bacterial backbone replication origins and selection markers are shown.

### EXAMPLES

The methods of the current technology are further illustrated by the following examples. These are provided by way of illustration and are not intended in any way to limit the scope of the disclosure.

### Example 1: PUC, and R6K Replication Origin Plasmid Replication and Production

pUC origin vector replication and production background: The vast majority of therapeutic plasmids use the pUC origin which is a high copy derivative of the pMB1 origin (closely related to the ColE1 origin). For pMB1 replication, plasmid DNA synthesis is unidirectional and does not require a plasmid bome initiator protein. The pUC origin is a copy up derivative of the pMB1 origin that deletes the accessory ROP (rom) protein and has an additional temperature sensitive mutation that destabilizes the RNAI/RNAII interaction. Shifting of a culture containing these origins from 30 to 42° C. leads to an increase in plasmid copy number. pUC plasmids can be produced in a multitude of E. coli cell lines.

RNA-OUT antibiotic free selectable marker background: Antibiotic-free selection is performed in E. coli strains containing phage lambda attachment site chromosomally integrated pCAH63-CAT RNA-IN-SacB (P5/6/6/6) as described in Williams, Supra, 2008. SacB (Bacillus subtilis levansucrase) is a counterselectable marker which is lethal to E. coli cells in the presence of sucrose. Translation of SacB from the RNA-IN-SacB transcript is inhibited by plasmid encoded RNA-OUT (FIG. 1B). This facilitates plasmid selection in the presence of sucrose, by inhibition of SacB mediated lethality.

R6K origin vector replication and production background: The R6K gamma plasmid replication origin requires a single plasmid replication protein 17 that binds as a replication initiating monomer to multiple repeated ‘iteron’ sites (seven core repeats containing TGAGNG consensus) and as a replication inhibiting dimer to repressive sites (TGAGNG) and to iterons with reduced affinity. Replication requires multiple host factors including IHF, DnaA, and primosomal assembly proteins DnaB, DnaC, DnaG (Abhyankar et al., 2003 J Biol Chem 278:45476-45484). The R6K core origin contains binding sites for DnaA and IHF that affect plasmid replication since n, IHF and DnaA interact to initiate replication.

Different versions of the R6K gamma replication origin have been utilized in various eukaryotic expression vectors, for example pCOR vectors (Soubrier et al., 1999, Gene Therapy 6:1482-88) and a CpG free version in pCpGfree vectors (Invivogen, San Diego CA), and pGM169 (University of Oxford). Incorporation of the R6K replication origin per se does not improve transgene expression levels compared to an optimized pUC origin vector (Soubrier et al., Supra, 1999). However, use of a conditional replication origin such as R6K gamma that requires a specialized cell line for propagation adds a safety margin since the vector will not replicate if transferred to a patient's endogenous flora.

A highly minimalized 6 iteron R6K gamma derived replication origin (SEQ ID NO: 1; FIG. 1E) that contains core sequences required for replication (including the DnaA box and stb 1-3 sites; Wu et al., 1995. J Bacteriol. 177: 6338-6345), but with the upstream π dimer repressor binding sites and downstream π promoter deleted (by removing one copy of the iterons) was described in Williams, Supra, 2014 and included herein by reference. This R6K origin contains 6 tandem direct repeat iterons (FIG. 1E). The NTC9385R Nanoplasmid™ vector including this minimalized R6K origin and the RNA-OUT AF selectable marker in the spacer region, was described in Williams, Supra, 2014 and included herein by reference.

Typical R6K production strains express from the genome the π protein derivative PIR116 that contains a P106L substitution that increases copy number (by reducing π dimerization; π monomers activate while π dimers repress). Fermentation results with pCOR (Soubrier et al., Supra, 1999) and pCpG plasmids (Hebel H L, Cai Y, Davies L A, Hyde S C, Pringle I A, Gill D R. 2008. Mol Ther 16: S110) were low, around 100 mg/L in PIR116 cell lines.

Mutagenesis of the pir-116 replication protein and selection for increased copy number has been used to make new production strains. For example, the TEX2pir42 strain contains a combination of P106L and P42L. The P42L mutation interferes with DNA looping replication repression. The TEX2pir42 cell line improved copy number and fermentation yield with pCOR plasmids with reported yields of 205 mg/L (Soubrier F. 2004. World Patent Application WO2004033664).

Other combinations of π copy number mutants that improve copy number include ‘P42L and P113S’ and ‘P42L, P106L and F107S’ (Abhyankar et al., 2004. J Biol Chem 279:6711-6719).

Williams, Supra, 2014 describes host strains expressing phage HK022 attachment site integrated pL promoter heat inducible π P42L, P106L and F107S high copy mutant replication (Rep) protein for selection and propagation of R6K origin Nanoplasmid™ vectors. This is an additional Nanoplasmid™ safety factor since R6K origin vectors can only replicate within the engineered Rep protein-expressing E. coli host strain.

RNA-OUT selectable marker-R6K plasmid propagation and fermentations described in Williams, Supra, 2014 were performed using heat inducible ‘P42L, P106L and F107S’ π copy number mutant cell lines such as DH5a host strain NTC711772=DH5α dcm-att_{λ}::P_{5/6 6/6}-RNA-IN-SacB, catR; att_{HK022}::pL (OL1-G to T) P42L-P106L-F107S (P3-), SpecR StrepR. Production yields up to 695 mg/L were reported.

Additional Cell Lines were Created and Disclosed Herein Including:
NTC821601 DH5α att_{λ}::P_{5/6 6/6}-RNA-IN-SacB, catR; att_{HK022}::pL (OL1-G to T) P42L-P106L-F107S (P3-), SpecR StrepR=dcm+ version of NTC711772
NTC940211 DH5α att_{λ}::P_{5/6 6/6}-RNA-IN-SacB, catR; att_{HK022}::pL (OL1-G to T) P42L-P106I-F107S P113S (P3-), SpecR StrepR=high copy substitution of P106I for P106L combined with P113S to create a quadruple copy number increasing mutant rep protein derivative of NTC821601
NTC1050811 DH5α att_{λ}::P_{5/5 6/6}-RNA-IN-SacB, catR; att_{HK022}::pL (OL1-G to T) P42L-P106I-F107S P113S (P3-), SpecR StrepR; att_{φ80}::pARA-CI857ts, tetR=pARA-CI857ts derivative of NTC940211. This strain contains a phage φ80 attachment site chromosomally integrated copy of a arabinose inducible C1857ts gene. Addition of arabinose to plates or media (e.g. to 0.2-0.4% final concentration) induces pARA mediated CI857ts repressor expression which reduces copy number at 30° C. through C1857ts mediated downregulation of the Rep protein expressing pL promoter [i.e. additional C1857ts mediates more effective downregulation of the pL (OL1-G to T) promoter at 30° C.]. Copy number induction after temperature shift to 37-42° C. is not impaired since the C1857ts repressor is inactivated at these elevated temperatures. A dcm-derivative (NTC1050811 dcm-) is used in cases where dcm methylation is undesirable.
NTC1011641; Stb14 attλ::P5/6 6/6-RNA-IN-SacB, catR; att_{HK022}::pL P42L-P106L-F107S (P3-) SpecR StrepR=Stb14 version of NTC661135 (XL1Blue-dcm-att_{λ}::P_{5/6 6/6}-RNA-IN-SacB, catR; att_{HK022}::pR pL P42L-P106L-F107S (P3-) SpecR StrepR described in Williams, Supra, 2014

Nanoplasmid™ production yields are improved with the quadruple mutant heat inducible pL (OL1-G to T) P42L-P106I-F107S P113S (P3-) compared to the triple mutant heat inducible pL (OL1-G to T) P42L-P106L-F107S (P3-) described in Williams, Supra, 2014. Yields in excess of 2 g/L Nanoplasmid™ have been obtained with the quadruple mutant NTC1050811 cell line (e.g. 2240 mg/L with NTC9 T7 A99 pA, Table 8)

Use of a conditional replication origin such as these R6K origins that requires a specialized cell line for propagation adds a safety margin since the vector will not replicate if transferred to a patient's endogenous flora.

### Example 2: PUC and R6K Origin Vector Production

Shake flask production: Shake flask production was performed using proprietary Plasmid+ shake culture medium. The seed cultures were started from glycerol stocks or colonies and streaked onto LB medium agar plates containing 50 μg/mL antibiotic (for ampR or kanR selection plasmids) or 6% sucrose (for RNA-OUT selection plasmids). The plates were grown at 30-32° C.; cells were resuspended in media and used to provide approximately 2.5 OD₆₀₀ inoculums for the 500 mL Plasmid+ shake flasks that contained 50 μg/mL antibiotic for ampR or kanR selection plasmids or 0.5% sucrose to select for RNA-OUT plasmids. Flask were grown with shaking to saturation at the growth temperatures as indicated in Tables 5, 6, 7, and 9.

Fermentation production: Fermentations were performed using proprietary fed-batch media (NTC3019, HyperGRO media) in New Brunswick BioFlo 110 bioreactors as described (Cames and Williams, Supra, 2011). The seed cultures were started from glycerol stocks or colonies and streaked onto LB medium agar plates containing 50 μg/mL antibiotic (for ampR or kanR selection plasmids) or 6% sucrose (for RNA-OUT selection plasmids). The plates were grown at 30-32° C.; cells were resuspended in media and used to provide approximately 0.1% inoculums for the fermentations that contained 50 μg/mL antibiotic for ampR or kanR selection plasmids or 0.5% sucrose for RNA-OUT plasmids. HyperGRO temperature shifts were as indicated in Tables 8 and 9.

Production hosts: pUC origin AmpR or KanR plasmid fermentations were performed in E. coli strain DH5α [F-Φ80lacZΔM15 Δ(lacZYA-argF) U169 recAl endA1 hsdR17 (rK-mK+)phoA supE44λ-thi-1 gyrA96 relA1](Invitrogen, Carlsbad CA) or Stb14.

Antibiotic-free pUC origin RNA-OUT plasmid fermentations were performed in E. coli strain DH5a containing phage lambda attachment site chromosomally integrated pCAH63-CAT RNA-IN-SacB (P5/6 6/6) as described in Williams, Supra, 2008. The production strain is NTC4862=DH5a attλ::P5/6 6/6-RNA-IN-SacB, catR.

Antibiotic-free R6K gamma origin RNA-OUT plasmid propagation and fermentations were performed using E. coli RNA-OUT selection hosts further encoding phage HK022 attachment site integrated pL promoter heat inducible π copy number mutant cell line lines, methods for the creation of which are described in Williams, Supra, 2014 and included herein by reference.

### Production Strains:

### pUC Origin-AmpR or KanR Antibiotic Selection Hosts

DH5α
Stb14

### pUC Origin-RNA-OUT Sucrose Selection Hosts

NTC4862 DH5α att_{λ}::P_{5/6 6/6}-RNA-IN-SacB, catR
NTC1011592 Stb14 attλ::P5/6 6/6-RNA-IN-SacB, catR

### R6K Origin-RNA-OUT Sucrose Selection Nanoplasmid™ Hosts

NTC1050811 DH5α att_{λ}::P_{5/6 6/6}-RNA-IN-SacB, catR; att_{HK022}::pL (OL1-G to T) P42L-P106I-F107S P113S (P3-), SpecR StrepR; att_{φ80}::pARA-CI857ts, tetR
NTC1011641 Stb14 attλ::P5/6 6/6-RNA-IN-SacB, catR; att_{HK022}::pL P42L-P106L-F107S (P3-) SpecR StrepR

Analytical Methods: Culture samples were taken at key points and at regular intervals during all fermentations. Samples were analyzed immediately for biomass (OD₆₀₀) and for plasmid yield. Plasmid yield was determined by quantification of plasmid obtained from Qiagen Spin Miniprep Kit preparations as described (Cames and Williams, Supra, 2011). Briefly, cells were alkaline lysed, clarified, plasmid was column purified, and eluted prior to quantification. Plasmid quality was determined by agarose gel electrophoresis analysis (AGE) and was performed on 0.8-1% Tris/acetate/EDTA (TAE) gels as described in Carnes and Williams, Supra, 2011.

### Example 3: PUC and R6K Origin Structured Vector Construction and Manufacturing

The R6K gamma origin (SEQ ID NO:1; FIG. 1E)-RNA-OUT (SEQ ID NO:5; FIG. 1B) bacterial replication-selection region (SEQ ID NO:8; FIG. 1C) was cloned into the polylinker region of a variety of pUC57 based vectors to create the pNTC-NP1, pNTC-NP2, pNTC-NP3, pNTC-NP4, pNTC-NP5, pNTC-NP6, pNTC-NP7, vectors. Each vector has different flanking restriction sites that can be used to retrofit a target vector to R6K replication-RNA-OUT selection. The 5′ and 3′ polylinker sequences flanking the R6K-RNA-OUT insert in the pNTC-NP 1-7 vectors are shown in Table 4. A pUC57 based version of the 1 CpG R6K gamma origin-2 CpG RNA-OUT bacterial replication-selection region (SEQ ID NO:9; FIG. 1D) was also created (pNTC-3×CpG NP1) and is shown in Table 4.

The R6K gamma origin (SEQ ID NO:1) is an engineered 6 iteron R6K origin (FIG. 1E). A pUC57 based version of a 7 iteron R6K gamma origin (SEQ ID NO:18; FIG. 1F)-RNA-OUT (SEQ ID NO:5; FIG. 1B) bacterial replication-selection region was also created and used to construct and evaluate the utility of additional iterons on manufacturing. Similarly, high quality, high yield manufacture was obtained with vectors differing only by containing either the SEQ ID NO:18 seven iteron R6K gamma origin or the six iteron R6K gamma origin (SEQ ID NO:1). For example, the following harvest production yields were obtained in 30-42° C. 10 hr ramp temperature shift HyperGRO fermentations:
SEQ ID NO:1 6 iteron 3203 bp R6K origin vector: a biomass of 120 OD₆₀₀; plasmid titer of 1363 mg/L; plasmid specific yield of 11.3 mg plasmid/L/OD₆₀₀
SEQ ID NO:18 7 iteron 3225 bp R6K origin vector: a biomass of 137 OD₆₀₀; plasmid titer of 1503 mg/L; plasmid specific yield of 11.0 mg plasmid/L/OD₆₀₀

The 7 iteron R6K gamma origin in SEQ ID NO:18 is a tandem duplication of iteron 5 (FIG. 1F; SEQ ID NO:18) but the 7 iteron R6K gamma origin vectors of the invention can be tandem duplications of any of the iterons given as SEQ ID NO:19; SEQ ID NO:20; SEQ ID NO:21; SEQ ID NO:22; SEQ ID NO:23 (FIG. 1E), or random combinations of SEQ ID NO:19; SEQ ID NO:20; SEQ ID NO:21; SEQ ID NO:22; SEQ ID NO:23 into 7 iteron R6K origin compositions, or iteron repeat variants that retain the TGAGNG consensus. Additional iteron derivatives (e.g. 8, 9 or 10 iteron vectors) are also contemplated for practice of the invention.

TABLE 4
pNTC multiple cloning site flanked R6K Origin-RNA-OUT selection marker vectors

| | R6K 5′ flanking | trpA | R6K | Linker | RNA OUT | RNA-OUT 3′ flanking |
|---|---|---|---|---|---|---|
| Vector | restriction sites | term | origin | site | Selection marker | restriction site |
| pNTC-NP1 | EcoRI, SacI, KpnI, | Yes | SEQ ID | DraIII^{a} | SEQ ID | NheI BamHI, XmaI, |
| (SEQ ID | NruI, NsiI, XmaIII, | | NO: 1 | | NO: 5 | ApaI, SalI, HincII, |
| NO: 10) | NotI, NheI | | | | | PstI, StuI, AatI, |
| | | | | | | SphI, HindIII (in R6K) |
| pNTC-NP2 | EcoRI, SacI, KpnI, | Yes | SEQ ID | DraIII^{a} | SEQ ID | SpeI, XmaI, SspI |
| (SEQ ID | NruI, NsiI, XmaIII, | | NO: 1 | | NO: 5 | BamHI, XmaI, ApaI, |
| NO: 11) | NotI, NheI | | | | | SalI, HincII, PstI, |
| | | | | | | StuI, AatI, SphI, |
| | | | | | | HindIII (in R6K) |
| pNTC-NP3 | EcoRI, SacI, KpnI, | Yes | SEQ ID | DraIII^{a} | SEQ ID | KpnI, SacI BamHI, |
| (SEQ ID | NruI, NsiI, XmaIII, | | NO: 1 | | NO: 5 | XmaI, ApaI, SalI, |
| NO: 12) | NotI, NheI | | | | | HincII, PstI, StuI, |
| | | | | | | AatI, SphI, HindIII |
| | | | | | | (in R6K) |
| pNTC-NP4 | NheI, XmaIII, NotI, | Yes | SEQ ID | DraIII^{a} | SEQ ID | EcoRI, SacI, KpnI |
| (SEQ ID | NsiI, NruI, KpnI, | | NO: 1 | | NO: 5 | |
| NO: 13) | SacI BamHI, XmaI, | | | | | |
| | ApaI, SalI, HincII, | | | | | |
| | SfcI, PstI, StuI, | | | | | |
| | AatI, SphI, HindIII | | | | | |
| | (in R6K) | | | | | |
| pNTC-NP5 | KasI, NheI | Yes | SEQ ID | DraIII^{a} | SEQ ID | KpnI AflIII PstI, |
| (SEQ ID | | | NO: 1 | | NO: 5 | AatI, SphI, HindIII |
| NO: 14) | | | | | | (in R6K) |
| pNTC-NP6 | EcoRI, PstI, EcoRV, | Yes | SEQ ID | DraIII^{a} | SEQ ID | KpnI, ApaI, PvuI, |
| (SEQ ID | BstXI, NotI, NheI | | NO: 1 | | NO: 5 | SalI, SacI |
| NO: 15) | | | | | | |
| pNTC-NP7 | BssHII PacI NheI | Yes | SEQ ID | DraIII^{a} | SEQ ID | KpnI PacI BssHII |
| (SEQ ID | | | NO: 1 | | NO: 5 | |
| NO: 16) | | | | | | |
| pNTC-3x | XhoI, XbaI, ApaI, | No | SEQ ID | BsrGI | SEQ ID | EcoRI, SacI, KpnI, |
| CpG NP1 | SalI, HincII, PstI, | | NO: 2 | | NO: 7 | NruI, NsiI, XmaIII, |
| (SEQ ID | StuI, AatI, SphI, | | | | | NotI, NheI, KpnI |
| NO: 17) | HindIII (in R6K) | | | | | |

^{a}Non-palindromic unique 3 bp NNN sticky end DraIII site (CACNNNGTG) separating R6K and RNA-OUT of sequence CACGTTGTG can be used to assemble R6K and RNA-OUT from separate pNTC vectors in directional multi-fragment ligation reactions

Viral and non-viral vector pUC origin-antibiotic selection bacterial backbone retrofits to R6K-RNA-OUT were performed by:
1) selecting restriction sites that flank the pUC origin and antibiotic selection marker region in the target viral and non-viral vector;
2) Identifying a pNTC-NP compatible polylinker-R6K-RNA-OUT polylinker cassette (either pNTC-NP1, 2, 3, 4, 5, 6, or 7; Table 4);
3) Excising the pUC origin antibiotic selection marker region and replacing with the selected R6K origin RNA-OUT region using the selected restriction digestion approach and standard ligase mediated cloning.

In some cases, the R6K origin and RNA-OUT units were assembled in multi-fragment ligations from separate restriction fragments using the non-palindromic DraIII linker site (see Table 4). In the case of the fd6 Ad helper retrofit (Table 9), a 3-fragment ligation was performed using a short 500 bp synthetic gene DraIII RNA-OUT-Ad helper-AvrII to link RNA-OUT to a unique AvrII site in the fd6 Ad helper eukaryotic region in a 12 kb AvrII-SalI restriction fragment, and to a SalI-R6K origin-DraIII fragment from pNTC-NP4.

Example vector maps and vector characteristics of the original pUC origin-antibiotic selection marker vector and the retrofitted R6K origin-RNA-OUT antibiotic free selection marker vectors are shown for Sleeping Beauty (FIG. 2; Table 6), AAV (FIG. 3; Table 7) and mRNA (FIG. 4; Table 8) vectors. The vector characteristics of the original pUC origin-antibiotic selection marker vector and the retrofitted R6K origin-RNA-OUT antibiotic free selection marker vectors are shown for AAV helper vectors (Table 8). The vector characteristics of pUC origin-RNA-OUT antibiotic free selection marker vector and the retrofitted R6K origin-RNA-OUT antibiotic free selection marker vectors are shown for Lentiviral vectors (Table 5) and AAV vectors (Table 7).

In all cases, the bacterial backbone size was <1 kb in the R6K origin-RNA-OUT antibiotic free selection marker retrofitted vectors (460-610 bp). This is well below the 1.1 kb bacterial backbone size limit required to improve vector expression level (Tables 1-2) and duration (Quiviger et al., Supra, 2014). In all cases, the original pUC origin-antibiotic selection bacterial backbone prior to retrofit was >1.2 kb (2340-2750 bp) as were the pUC origin-RNA-OUT retrofits (1210-1500 bp). Thus, these AAV, AAV helper, Sleeping Beauty, and Lentiviral R6K origin-RNA-OUT antibiotic free selection marker retrofit vectors meet the short spacer region requirement for improved expression level and duration compared to the original pUC origin-antibiotic selection marker vector. Additionally, these AAV, AAV helper, Sleeping Beauty, and Lentiviral R6K origin-RNA-OUT antibiotic free selection marker retrofit vectors have no chance of antibiotic marker gene transfer by transduction (AAV, Lentiviral vectors) or transposition (Sleeping Beauty vectors) due to removal of the KanR or ampR antibiotic resistance selection marker in the parent vector. Additionally, the vectors of the current technology do not require the complicated difficult to scale expensive additional manufacturing steps required to remove the large bacterial region between the eukaryotic polyA and promoter with minicircle vectors (Kay et al., Supra, 2010).

However, in a Lentiviral vector the eukaryotic region contains flanking direct repeat LTRs, in an AAV vector the eukaryotic region contains flanking inverted terminal repeats, while in a Sleeping Beauty transposon vector the eukaryotic region contains flanking transposon IR/DR termini. These flanking sequences are all structured DNA sequences.

Levy, Supra, 2004 teaches that replication intermediates form when any high copy number prokaryotic origin of replication is <1 kb from a structured DNA sequence such as an enhancer, LTR or IRES, but not when the high copy replication origin is >1.5 kb. Consistent with this, Replication intermediates were formed in all pUC origin-RNA-OUT marker vectors in which the pUC origin was <1 kb from a Lentiviral vector LTR (Table 5: 400 bp) or a pUC origin-antibiotic resistance marker vector in which the pUC origin was <1 kb from a Sleeping Beauty IR/DR (Table 6; 280 bp). For AAV and mRNA vectors, the original pUC origin-antibiotic selection marker vectors have the pUC origin 0 bp from an ITR (AAV vector; Table 7) or 170 bp from a A99 repeat (mRNA vector, Table 8) which may make a replication intermediate that is too small to detect on an agarose gel. However, in these cases production yields were very low, indicative of low plasmid copy number due to replication blockage. By contrast, as expected, in the case where the original pUC origin-antibiotic selection marker vector pUC origin was >1.5 kb from a structured DNA sequence (A60 repeat), high plasmid production yields were obtained (Table 8: mRNA vector pGEM4Z T7 A60).

Williams, Supra, 2017 reported that pUC origin vector production yield is improved with a PAS-BH extended pUC origin when the pUC origin is >1.5 kb from a homopolymeric A64C31 repeat. However, production yields were low when a PAS-BH extended pUC origin is orientated <400 bp from the A64C31 repeat (Table 8, see footnotes d and e). This teaches that addition of a PAS-BH primosomal assembly site does not overcome the poor pUC origin directed replication of closely positioned structured DNA sequences.

Since the pUC origin itself is 1 kb, there is no configuration to make a <1.1 kb bacterial region AAV, Lentiviral, Retroviral or transposon vector containing the pUC origin which is not predicted to produce replication intermediates as seen above and predicted by Levy, Supra, 2004 and poor plasmid yields as reported herein.

Surprisingly, replication intermediates were not observed in any R6K origin-RNA-OUT antibiotic free selection marker retrofitted vectors, include those in which the R6K origin was <1 kb from a Lentiviral vector LTR (Table 5: 400 bp) or a Sleeping Beauty IR/DR (Table 6; <40 bp). Further, for AAV vectors, while the original pUC origin-antibiotic selection marker vectors with the pUC origin 0 bp from the ITR had very poor production yields, the two R6K origin-RNA-OUT antibiotic free selection marker retrofitted vectors with the R6K origin 40 bp from the ITR had much higher production yields (Table 7). This improved production is specific to R6K and not RNA-OUT, since the two AAV pUC-RNA-OUT retrofits with the pUC origin 50 bp from the ITR had equally poor plasmid production yields as the original pUC antibiotic marker vector (Table 7); as well the direct comparison of pUC-RNA-OUT with R6K-RNA-OUT retrofits positioned 400 bp from an LTR repeat in a Lentiviral backbone showed replication intermediates with all three pUC-RNA-OUT backbones but none of the three R6K-RNA-OUT backbones (Table 5).

This surprising improvement in plasmid copy number (plasmid production yields) and quality (eliminated replication intermediates) with the R6K origin vector implies that the R6K origin can replicate through a structured DNA sequence more effectively than the pUC origin. While Levy, Supra, 2004 teaches that replication intermediates form when any high copy number prokaryotic origin of replication is <1 kb from a structured DNA sequence such as an enhancer, LTR or IRES, but not when the high copy replication origin is >1.5 kb away, the examples provided by Levy, Supra, 2004 were all with pUC origin plasmids.

A fundamental difference between these replication origins is that the pUC origin is a Pol I dependent origin of replication while the R6K origin is a Pol III dependent origin of replication. With the pUC origin the RNAII primer forms an RNA: DNA R-loop that is cleaved by RNase H to create a primer for DNA Pol I directed DNA synthesis during initial leading strand synthesis. DNA synthesis then converts from slow DNA Pol I to the highly processive DNA Pol III from 400 bp to up to 1.3 kb downstream of the origin (Allen et al., 2011. Nucleic Acids Research 39:7020-33). The R6K gamma replication origin rep protein interacts with dnaB helicase and dnaG primase which creates short RNA primers for DNA Pol III replication without requirement for DNA Pol I (Abhyankar et al., Supra, 2003). The pUC origin DNA Pol I replication zone of up to 1.3 kb from the origin corresponds closely with the Levy, Supra, 2004 defined upper limit of replication intermediate formation (between 1 and 1.5 kb from the origin). We propose that the observed surprisingly improved replication of structured DNA when in close proximity to the R6K but not the pUC origin is due to an unexpected improvement of replication of structured DNA sequences by DNA Pol III compared to DNA Poll.

The vector methods and compositions disclosed herein demonstrate that a Pol III-dependent origin of replication such as the R6K origin can be used to replicate structured DNA sequences which are poorly replicated by a Pol I-dependent origin of replication such as the pUC origin.

These results demonstrate the vectors of the invention are useful for improving viral and non-viral vector manufacturing yield and quality.

### Example 4: Improved Performance of R6K Origin Structured Vector

The vectors of the invention are additionally useful for eliminating antibiotic resistance marker gene transfer by viral and non-viral vectors; reducing transfection associated toxicity; improving transposition from non-viral transposon vectors; improving packaging titers from viral vectors; improving expression of viral and non-viral vector encoded transgenes, etc.

As an example, R6K origin third generation lentiviral vectors [4 vectors: Table 5 transfer plasmid, gag pol packaging plasmid; env plasmid; REV plasmid (not shown) with R6K origin and <1 kb bacterial backbone] of the invention showed reduced toxicity and improved viral packaging titers compared to pUC origin vector comparators with >1.5 kb bacterial backbone. Transfection of Lenti-X 293 T cell line (Takara Bio Mountain View, CA) with Table 5 R6K origin third generation lentiviral vectors with <1 kb bacterial backbone or original pUC origin-antibiotic selection marker vector with >1.5 kb bacterial backbone control in 24 well plates using Lipofectamine 3000 (Thermo Fisher Scientific, Waltham, MA) as recommended by the manufacturer resulted in higher titer lentivirus production (>1.5 kb bacterial backbone pUC origin control vectors: 1.00×±0.32; <1 kb bacterial backbone R6K origin vectors 1.45×±0.42) as measured using the Lenti-X p24 Rapid Titer Kit (Takara Bio Mountain View, CA). Transfection of Lenti-X 293 T cell line in 24 well plates with Table 5 third generation lentiviral vectors (>1.5 kb bacterial backbone pUC origin control or <1 kb bacterial backbone R6K origin) using Calcium Phosphate transfection as described (Marino M P, Luce M J, Reiser J. 2003, Methods Mol Biol 229:43-55) resulted in higher titer lentivirus production (>1.5 kb bacterial backbone pUC origin control: 1.00×±0.30; <1 kb bacterial backbone R6K origin vectors: 1.32×±0.19) as measured using the Lenti-X p24 Rapid Titer Kit (Takara Bio Mountain View, CA). Significantly, Calcium Phosphate transfection of the >1.5 kb bacterial backbone pUC origin third generation Lentiviral vectors resulted in extensive transfection associated toxicity (>80% cell death) compared to low toxicity with the R6K origin <1 kb bacterial backbone R6K origin third generation Lentiviral vectors in this 24 well plate transfection. This reduced transfection associated toxicity should result in dramatically improved viral titers in larger manufacturing scale transfections. These results demonstrate the <1 kb bacterial backbone R6K origin Nanoplasmid vectors of the invention reduce transfection associated toxicity and improve packaging titers from viral vectors compared to >1.5 kb bacterial backbone vectors.

### SUMMARY

While the above description contains many examples, these should not be construed as limitations on the scope of the disclosure, but rather should be viewed as an exemplification of preferred embodiments thereof. Many other variations are possible.

For example, in the vectors of the current technology various orientations of the Pol III dependent replication origin, and the RNA selectable marker, may be utilized. For example, any of the eight orientations of the Pol III dependent replication origin, and the RNA selectable marker in vectors of the current technology may be used (i.e. ←Pol III replication origin RSM→; ←Pol III replication origin ←RSM; Pol III replication origin→RSM→; Pol III replication origin→←RSM; ←RSM Pol III replication origin→; ←RSM←Pol III replication origin; RSM→Pol III replication origin→; RSM→←Pol III replication origin).

Further, a variety of RNA selectable markers know in the art may be substituted for RNA-OUT.

Further, an antibiotic resistance maker may be substituted for RNA-OUT, for example in the case where a simple retrofit of the pUC origin to the R6K origin is desired to improve plasmid production yield and or quality.

Thus, the reader will see that the improved Pol III dependent replication origin vectors of the current technology provide for an approach to reduce transfection associated toxicity, improve transposition from non-viral transposon vectors, improve packaging titers from viral vectors, improve expression of viral and non-viral vector encoded genes, and eliminate viral vector and non-viral vector mediated antibiotic selection marker gene transfer (i.e. through incorporation of a bacterial region preferably less than 1000 bp) while dramatically improving manufacture compared to alterative vectors such as pUC plasmids and minicircles.

Accordingly, the scope of the disclosure should be determined not by the embodiments illustrated, but by the appended claims.

TABLE 5
Lentiviral vectors: pUC origin-RNA-OUT versus R6K origin-RNA-OUT shake flask production yields/quality

| | | | Transfer | | | | | | | | |
|---|---|---|---|---|---|---|---|---|---|---|---|
| | | | plasmid | Replication | | | | | Harvest | | |
| | | | bacterial | origin | Replication | | | | spec | | |
| | | | backbone | spacing | origin | | | | yield | Harvest | |
| Lentiviral | Replication | Vector | (LTR | from SV40 | spacing | Cell | Production | Harvest | (mg/L/ | yield | Production |
| Vector | origin | size | spacing)^{a} | Ori^{b} | front LTR^{c} | line^{d} | culture^{e} | OD₆₀₀ | OD₆₀₀) | (mg/L) | quality^{f} |
| Transfer | pUC origin | 9.9 kb | 1210 bp | <50 bp | 400 bp | DH5α | 30 to 37 C. | 7.5 | 3.4 | 26 | CCC |
| plasmid 1 | | | (1210 bp) | | | | | | | | Monomer + |
| with SV40 | | | | | | | | | | | Replicate |
| Ori | | | | | | | | | | | Intermediate |
| | R6K origin | 9.2 kb | 460 bp | <50 bp | 400 bp | DH5α | 30 to 37 C. | 5.0 | 4.7 | 24 | CCC |
| | | | (460 bp) | | | | | | | | Monomer |
| Transfer | pUC origin | 7.3 kb | 1210 bp | <50 bp | 400 bp | DH5α | 30 to 37 C. | 9.0 | 3.9 | 35 | CCC |
| plasmid 2 | | | (1210 bp) | | | | | | | | Monomer + |
| with SV40 | | | | | | | | | | | Replicate |
| Ori | | | | | | | | | | | Intermediate |
| | R6K origin | 6.5 kb | 460 bp | <50 bp | 400 bp | DH5α | 30 to 37 C. | 8.4 | 7.2 | 61 | CCC |
| | | | (460 bp) | | | | | | | | Monomer |
| Transfer | pUC origin | 8.3 kb | 1210 bp | <50 bp | 400 bp | DH5α | 30 to 37 C. | 16.8 | 3.0 | 51 | CCC |
| plasmid 3 | | | (1210 bp) | | | | | | | | Monomer + |
| with SV40 | | | | | | | | | | | Replicate |
| Ori | | | | | | | | | | | Intermediate |
| | R6K origin | 7.5 kb | 460 bp | <50 bp | 400 bp | DH5α | 30 to 37 C. | 11.9 | 7.7 | 92 | CCC |
| | | | (460 bp) | | | | | | | | Monomer |
| Gag pol | pUC origin | 8.4 kb | 1210 bp | NA | NA | DH5α | 30 to 37 C. | 10.2 | 4.3 | 44 | CCC |
| packaging | | | | | | | | | | | Monomer |
| plasmid | R6K origin | 7.5 kb | 460 bp | NA | NA | DH5α | 30 to 37 C. | 9.7 | 7.6 | 73 | CCC |
| | | | | | | | | | | | Monomer |
| Envelope | pUC origin | 5.4 kb | 1210 bp | NA | NA | DH5α | 30 to 37 C. | 17.0 | 5.7 | 96 | CCC |
| plasmid | | | | | | | | | | | Monomer |
| | R6K origin | 4.4 kb | 460 bp | NA | NA | DH5α | 30 to 37 C. | 10.8 | 6.0 | 65 | CCC |
| | | | | | | | | | | | Monomer |

^{a}All vectors are antibiotic free RNA-OUT selection retrofits. Original pUC origin-antibiotic selection marker vector bacterial backbone size were all >1.5 kb
^{b}Distance in bp from 3′ end of replication origin to SV40 origin in direction of replication
^{c}Distance in bp from 3′ end of replication origin to nearest LTR in direction of replication
^{d}DH5α = NTC4862 for pUC origin, NTC1050811 for R6K origin
^{e}500 mL Plasmid + Shake Flask Culture temperature shifted from 30 to 37 C.
^{f}Harvest plasmid preparations with detectable replication intermediates indicated. Levy, Supra, 2004 teach that replication intermediates form when any high copy number prokaryotic origin of replication is less than 1000 bp from a promoter/enhancer or LTR promoter or DNA repeat or a complex secondary structure such as an IRES, while replication intermediates do not form if the origin of replication is a distance greater than about 1.5 kb.

TABLE 6
Sleeping Beauty Transposon and transposase vector: pUC versus R6K origin shake flask production yields/quality

| | | | | bacterial | Replica- | | | | | Harvest | | |
|---|---|---|---|---|---|---|---|---|---|---|---|---|
| Replica- | | Vector | | backbone | tion | | | | | spec | | |
| tion | | IR/DR | Vec- | size | origin | | | Produc- | | yield | Harvest | Produc- |
| origin/ | Bacterial | Configura- | tor | (IR/DR | spacing from | | Cell | tion | Harvest | (mg/L/ | yield | tion |
| vector | selection | tion | size | spacing) | IR/DR^{a} | | line^{b} | culture^{c} | OD₆₀₀ | OD₆₀₀) | (mg/L) | quality^{d} |
| Transposon | KanR | IR DR(R) | 5.5 kb | 2600 bp | 280 | bp | DH5α | 30 to | 1.9 | 2.1 | 4 | CCC |
| vector | | <pUC<kanR | | (2600 bp) | | | | 37 C. | | | | Monomer + |
| pUC origin | | IR DR(L) | | | | | | | | | | Replicate |
| (pUC57-kan- | | Promoter> | | | | | | | | | | Inter- |
| SB1: see | | Transgene> | | | | | | | | | | mediate |
| FIG. 2) | | | | | | | | | | | | |
| Transposon | RNA- | IR DR(R) < | 3.4 kb | 475 bp | <40 | bp | DH5α | 30 to | 11.2 | 1.0 | 11 | CCC |
| vector | OUT AF | R6K R-OUT > | | (475 bp) | | | | 37 C. | | | | Monomer |
| R6K origin | | IR DR(L) | | | | | | | | | | |
| (NTC9-SB1: | | Promoter> | | | | | | | | | | |
| see FIG. 2) | | Transgene> | | | | | | | | | | |
| Transposon | RNA- | IR DR(R) < | 3.6 kb | 475 bp | <40 | bp | DH5α | 30 to | 4.1 | 6.2 | 25 | CCC |
| vector | OUT AF | R6K R-OUT > | | (475 bp) | | | | 37 C. | | | | Monomer |
| R6K origin | | IR DR(L) | | | | | | | | | | |
| (NTC9-SB2) | | Promoter> | | | | | | | | | | |
| | | Transgene> | | | | | | | | | | |

| | | | | | | | | | | | |
|---|---|---|---|---|---|---|---|---|---|---|---|
| Transposase | KanR | Not | 4.7 kb | 2600 bp | Not | DH5α | 30 to | 21.6 | 1.8 | 39 | CCC |
| vector | | Applicable | | (NA) | Applicable | | 37 C. | | | | Monomer |
| pUC origin | | | | | | | | | | | |
| Transposase | RNA- | Not | 2.5 kb | 475 bp | Not | DH5α | 30 to | 10.9 | 4.7 | 51 | CCC |
| vector | OUT AF | Applicable | | (NA) | Applicable | | 37 C. | | | | Monomer |
| R6K origin | | | | | | | | | | | |

^{a}Distance in bp from 3′ end of replication origin to nearest Sleeping Beauty IR/DR in direction of replication
^{b}DH5α = DH5α for pUC57-kanR pUC origin, NTC1050811 for R6K origin
^{c}500 mL Plasmid + Shake Flask Culture temperature shifted from 30 to 37 C.
^{d}Harvest plasmid preparations with detectable replication intermediates indicated. Levy, Supra, 2004 teach that replication intermediates form when any high copy number prokaryotic origin of replication is less than 1000 bp from a promoter/enhancer or LTR promoter or DNA repeat or a complex secondary structure such as an IRES, while replication intermediates do not form if the origin of replication is a distance greater than about 1.5 kb.

TABLE 7
AAV vectors: pUC versus R6K origin shake flask production yields/quality

| | | | | | | | Replica- | | | | Har- | | |
|---|---|---|---|---|---|---|---|---|---|---|---|---|---|
| | | | | | | | tion | | Pro- | | vest | | Pro- |
| | | | | | bacterial | | origin | | duc- | | spec | Harv- | duc- |
| | Replica- | | Vector | | backbone | | spacing | | tion | Har- | yield | est | tion |
| AAV | tion | Bacterial | Configura- | Vector | size (ITR | Bacterial | from | Cell | cul- | vest | (mg/L/ | yield | quali- |
| Vector | origin | selection | tion | size | spacing)^{e} | selection | ITR^{a} | line^{b} | ture^{c} | OD₆₀₀ | OD₆₀₀) | (mg/L) | ty^{d} |
| Back- | pUC | AmpR | ITR <F1Ori- | 5.4 kb | 2600 bp | AmpR | 0 bp | Stbl4 | 30-37 C. | 17.8 | 0.4 | 8 | CCC |
| bone 1^{e} | origin | | AmpR>pUC> | | (2600 bp) | | | | shake | | | | Mono- |
| | (pAAV: | | ITR CMV- | | | | | | | | | | mer |
| | see | | transgene- | | | | | | | | | | |
| | FIG. 3) | | pA | | | | | | | | | | |
| | pUC | RNA- | ITR <pUC | 4.0 kb | 1240 bp | RNA- | 50 bp | Stbl4 | 30-37 C. | 10.4 | 0.7 | 7 | CCC |
| | origin | OUT AF | R-OUT> | | (1240 bp) | OUT AF | | | shake | | | | Mono- |
| | (NTC8- | | ITR CMV- | | | | | | | | | | mer |
| | AAV: | | transgene- | | | | | | | | | | |
| | see | | pA | | | | | | | | | | |
| | FIG. 3) | | | | | | | | | | | | |
| | R6K | RNA- | ITR <R6K | 3.3 kb | 490 bp | RNA- | 40 bp | Stbl4 | 30-37 C. | 11.8 | 3.5 | 42 | CCC |
| | origin | OUT AF | R-OUT> | | (490 bp) | OUT AF | | | shake | | | | Mono- |
| | (NTC9- | | ITR CMV- | | | | | | | | | | mer |
| | AAV: | | transgene- | | | | | | | | | | |
| | see | | pA | | | | | | | | | | |
| | FIG. 3) | | | | | | | | | | | | |
| | R6K | RNA- | ITR <R6K | 7.7 kb | 490 bp | RNA- | 40 bp | Stbl4 | 30-37 C. | 11.0 | 2.1 | 23 | CCC |
| | origin | OUT AF | R-OUT> | | (4940 bp) | OUT AF | | | shake | | | | Mono- |
| | | | Rep> Cap> | | | | | | | | | | mer |
| | | | ITR CMV- | | | | | | | | | | |
| | | | transgene- | | | | | | | | | | |
| | | | pA | | | | | | | | | | |
| Back- | pUC | RNA- | ITR <pUC | 5.5 kb | 1250 bp | RNA- | 50 bp | Stbl4 | 30-37 C. | 11.7 | 0.4 | 5 | CCC |
| bone 2 | origin | OUT AF | R-OUT> | | (NA) | OUT AF | | | shake | | | | Mono- |
| | (NTC8) | | ITR CAG- | | | | | | | | | | mer |
| | | | transgene- | | | | | | | | | | |
| | | | WPRE-pA | | | | | | | | | | |
| | R6K | RNA- | ITR <R6K | 4.8 kb | 490 bp | RNA- | 40 bp | Stbl4 | 30-37 C. | 11.1 | 1.6 | 18 | CCC |
| | origin | OUT AF | R-OUT> | | (NA) | OUT AF | | | shake | | | | Mono- |
| | (NTC9) | | ITR CAG- | | | | | | | | | | mer |
| | | | transgene- | | | | | | | | | | |
| | | | WPRE-pA | | | | | | | | | | |

^{a} Distance in bp from 3′ end of replication origin from nearest AAV ITR in direction of replication
^{b}Stbl4 = Stbl4 for pUC origin-ampR, NTC1011592 for pUC origin-RNA-OUT, NTC1011641 for R6K origin-RNA-OUT
^{c}Plasmid + Shake Flask Culture temperature shifted from 30 to 37 C.
^{d}High quality CCC monomer with no detectable replication intermediate and low amounts of recombination mediated ITR deletion. May not see replication intermediates on gel with pUC origin since <50 from DNA structure, but probably formed since plasmid yield lower than R6K origin
^{e}A R6K origin-RNA-OUT MIP intron version of this vector with 6 bp ITR spacing was unclonable, presumably due to toxicity of juxtaposing the 2 AAV ITR immediately adjacent to each other. A R6K origin-RNA-OUT MIP intron version in a second AAV vector backbone (10 bp ITR spacing) was also unclonable

TABLE 8
mRNA vector: pUC versus R6K origin DH5α^{f} HyperGRO ^{g} Fermentation Yields/quality

| | | | | | | | Replica- | | | Harvest | Harvest | | | |
|---|---|---|---|---|---|---|---|---|---|---|---|---|---|---|
| polyA | | | | | | | tion | | | spec | spec | | | |
| mRNA | Replica- | | Vector | | Bacterial | | origin | | | yield | yield | Harvest | Harvest | Produc- |
| expression | tion | Selec- | Configura- | Vector | backbone | | bp from | | Harvest | (mg/L/ | (mg/ | yield | yield | tion |
| vector | origin | tion | tion | size | size | | polyA | | OD₆₀₀ | OD₆₀₀) | gWCW) | (mg/L) | (g)^{b} | quality |
| pT3/T7 | pUC origin | AmpR | A99<pUC< | 7.2 kb | 2660 | bp | 170 | bp | 113 | 3.8 | 1.6 | 430^{a} | 43 | CCC |
| A99 pA^{h} | (pT3/T7 | | AmpR T7> | | | | | | | | | | | Monomerⁱ |
| mRNA | A99 pA) | | Transgene | | | | | | | | | | | |
| expression | pUC origin + | kanR | A99<KanR | 7.2 kb | 2660 | bp | 5000 | bp | 140 | 8.9 | 4.1 | 1250^{a} | 12.5 | CCC |
| vector and | PAS-BH^{d} | | pUC>PAS- | | | | | | | | | | | Monomer |
| retrofit | (NTC7 T7 | | BH> T7> | | | | | | | | | | | |
| derivatives | A99 pA) | | Transgene | | | | | | | | | | | |
| | R6K origin | RNA- | A99<R-OUT | 5.2 kb | 610 | bp | 4600 | bp | 147 | 15.3 | 6.7 | 2240^{a} | 22.4 | CCC |
| | (NTC9 T7 | OUT AF | R6K> T7> | | | | | | | | | | | Monomer |
| | A99 pA) | AmpR | Transgene | | | | | | | | | | | |
| pGEM4Z | pUC origin | | A60 AmpR> | 6.8 kb | 2500 | bp | 4400 | bp | 112 | 8.3 | 4.4 | 929 | 9.3 | CCC |
| A60 pA^{c} | (pGEM4Z | | pUC> T7> | | | | | | | | | | | Monomer |
| mRNA | T7 A60 pA) | | Transgene | | | | | | | | | | | |
| expression | pUC origin + | RNA- | A60<R-OUT | 5.9 kb | 1500 | bp | 4600 | bp | 146 | 11.5 | 4.9 | 1673^{a} | 16.7 | CCC |
| vector and | PAS-BH^{e} | OUT AF | pUC> PAS- | | | | | | | | | | | Monomer |
| retrofit | (NTC8 T7 | | BH> T7> | | | | | | | | | | | |
| derivatives | A60 pA) | | Transgene | | | | | | | | | | | |
| | R6K origin | RNA- | A60<R-OUT | 4.9 kb | 460 | bP | 4300 | bp | 113 | 13.1 | 5.0 | 1483^{a} | 14.8 | CCC |
| | (NTC9 T7 | OUT AF | R6K> T7> | | | | | | | | | | | Monomer |
| | A60 pA) | | Transgene | | | | | | | | | | | |

^{a}PolyA region correct by sequencing
^{b}gram plasmid/10 L fermentor
^{c}See FIG. 4 for vector maps
^{d}KanR-pUC + PASBH vector backbone described in Williams 2017 WO2017025447. This pUC + PASBH backbone dramatically improved plasmid yield when oriented >1.5 kb away from polyA64-polyC31 compared to a pUC origin oriented <200 bp away from the polyA64-polyC31 repeat or a pUC + PASBH origin oriented <400 bp away from the polyA64-polyC31 repeat (Williams, Supra, 2017, P1140-K2 versus P1140 and P1140-K1, Table 1). Thus PAS-BH does not dramatically improve pUC origin replication into a repeat structure <400 bp from the pUC origin.
^{e}RNA-OUT-pUC + PASBH vector backbone described in Williams, Supra, 2017. This pUC + PASBH backbone dramatically improved plasmid yield when oriented >1.5 kb away from polyA64-polyC31 compared to a pUC origin oriented <200 bp away from the polyA64-polyC31 repeat or a pUC + PASBH origin oriented <400 bp away from the polyA64-polyC31 repeat. (Williams, Supra, 2017, Table 1: P1140-AF2 versus P1140 and P1140-AF1). Thus PAS-BH does not dramatically improve pUC origin replication into a repeat structure <400 bp from the pUC origin.
^{f}DH5α = NTC4862 for NTC8; NTC1050811 for NTC9: NTC1050811 DH5α att_{λ}::P_{5/6 6/6}-RNA-IN-SacB, catR; att_{HK022}::pL (OL1-G to T) P42L-P106I-F107S P113S (P3−), SpecR StrepR; att_{φ80}::pARA-CI857ts, TetR. Arabinose induces pARA-CI857ts expression which reduces copy number through CI857ts mediated downregulation of the Rep protein expressing pL promoter
^{g}HyperGRO fermentation 30-42° C. temperature shift at 55 OD₆₀₀. For NTC9 vectors fermentation media contained 0.2% arabinose
^{h}Elango et al., 2005. Biochemical and Biophysical Research Communications 330: 958-66 teach polyA100 mRNA vectors are unstable in DH5α and XL1Blue and cannot be produced in mg quantities. See FIG. 4 for vector maps
ⁱMay not see replication intermediates on gel with pUC origin since <170 bp from DNA structure, but probably formed since plasmid yield lower than with R6K origin vectors

TABLE 9
AAV helper vectors: pUC versus R6K origin plasmid production yields/quality

| | | | | | | | | | | Harvest | | |
|---|---|---|---|---|---|---|---|---|---|---|---|---|
| | | | | | | | | | | spec | | |
| AAV | | | | | bacterial | | | | | yield | Harvest | |
| Helper | Replication | | Vector | | backbone | | Cell | Production | Harvest | (mg/L/ | yield | Production |
| Vector | origin | Selection | size | | size | | line^{b} | culture ^{a} | OD₆₀₀ | OD₆₀₀) | (mg/L) | quality |
| AAV2 Rep/ | pUC origin | RNA- | 5.7 | kb | 1220 | bp | DH5α | 30 to 37 C.^{a} | 16.5 | 2.6 | 43 | CCC |
| AAV9 Cap | | OUT AF | | | | | | | | | | Monomer |
| (Rep Cap | | | | | | | | | | | | |
| helper) | | | | | | | | | | | | |
| | R6K origin | RNA- | 4.9 | kb | 470 | bp | DH5α | 30 to 37 C.^{a} | 17.5 | 5.5 | 96 | CCC |
| | | OUT AF | | | | | | | | | | Monomer |
| Fd6 | pUC origin | ampR | 15.4 | kb | 2750 | bp | Stbl4^{c} | 30 C.^{a,c} | 20 | 0.8^{d} | 16^{d} | CCC |
| (Ad Helper) | | | | | | | | | | | | Monomer |
| | | | | | | | | | | | | and deletion |
| | | | | | | | | | | | | productions |
| | R6K origin | RNA- | 12.9 | kb | 540 | bp | DH5α | 30 to 37 C.^{a} | 17.8 | 1.3 | 23 | CCC |
| | | OUT AF | | | | | | | | | | Monomer |
| pHelper Rep | R6K origin | RNA- | 14.2 | kb | 470 | bp | DH5α | 30 to 37 C.^{a} | 6.8 | 4.8 | 33 | CCC |
| Cap NP (Ad | | OUT AF | | | | | | | | | | Monomer |
| Helper + Rep | | | | | | | | | | | | |
| Cap single | | | | | | | | | | | | |
| helper plasmid) | | | | | | | | | | | | |
| pHelper | pUC origin | ampR | 11.6 | kb | 2340 | bp | DH5α | 30-42 C. | 129 | 14.2 | 1833 | CCC |
| (Ad Helper) | | | | | | | | HyperGRO | | | | Monomer |
| | | | | | | | | shift | | | | |
| pHelper-NP | R6K origin | RNA- | 9.8 | kb | 460 | bp | DH5α | 30-42 C. | 84.5 | 18.1 | 1530 | CCC |
| (Ad Helper) | | OUT AF | | | | | | HyperGRO | | | | Monomer |
| | | | | | | | | RAMP^{e} | | | | |

^{a}500 mL Plasmid + Shake Flask Culture;
^{b}DH5α = NTC1050811 for NTC9: NTC1050811 DH5α att_{λ}::P_{5/6 6/6}-RNA-IN-SacB, catR; att_{HK022}::pL (OL1-G to T) P42L-P106I-F107S P113S (P3−), SpecR StrepR; att_{φ80}::pARA-CI857ts, TetR. Arabinose induces pARA-CI857ts expression which reduces copy number through CI857ts mediated downregulation of the Rep protein expressing pL promoter. Retrofits were obtained using Sucrose + 0.2% Arabinose plates to reduce copy number
^{c}Fd6 Stbl4 best cell line, Fd6/Stbl3 see deletions, Fd6/DH5α and Fd6/XL1Blue cell lines are unstable, used 30 C. throughout since plasmid lost in 30-37 C. process
^{d}Actual yield lower due to high gDNA in minipreps
^{e}HyperGRO fermentation, inoculated from sucrose + 0.2% arabinose culture grown at 30° C., arabinose added to batch medium to 0.2% final concentration. 30-42° C. ramp temperature shift

1-18. (canceled)

19. An engineered E. coli cell comprising one or more copies of a chromosomally-integrated arabinose-inducible CI857ts gene.

20. The engineered E. coli cell of claim 19 further comprising a mutated Rep protein, wherein the mutated Rep protein comprises the following mutations: P42L; P106L; F1075; and P113S.

21. The engineered E. coli cell of claim 20 further comprising a covalently closed circular plasmid, the covalently closed circular plasmid comprising a backbone comprising a Pol III-dependent R6K origin of replication and an RNA-OUT RNA selectable marker, wherein the backbone is less than 1000 bp.

22. The engineered E. coli cell of claim 19 further comprising a covalently closed circular plasmid, the covalently closed circular plasmid comprising a backbone comprising a Pol III-dependent R6K origin of replication and an RNA-OUT RNA selectable marker, wherein the backbone is less than 1000 bp.

23. The engineered E. coli cell of claim 22, wherein the Pol III-dependent R6K origin of replication possesses at least 95% sequence identity to a sequence selected from the group consisting of SEQ ID NO: 1, SEQ ID NO: 2, SEQ ID NO: 3, SEQ ID NO: 4, and SEQ ID NO: 18.

24. The engineered E. coli cell of claim 22, wherein the RNA-OUT RNA selectable marker is an RNA-IN regulating RNA-OUT functional variant with at least 95% sequence identity to a sequence selected from the group consisting of SEQ ID NO: 5, and SEQ ID NO: 7.

25-36. (canceled)
